(19) United States
(12) Patent Application Publication
(10) Pub. No.: US 2023/0312478 A1
(43) Pub. Date: Oct. 5, 2023

(54) HERBICIDAL COMPOUNDS

(71) Applicant: SYNGENTA CROP PROTECTION AG, Basel (CH)

(72) Inventors: James Alan MORRIS, Bracknell, Berkshire (GB); Sally Elizabeth RUSSELL, Bracknell, Berkshire (GB)

(73) Assignee: SYNGENTA CROP PROTECTION AG, Basel (CH)

(21) Appl. No.: 18/040,462

(22) PCT Filed: Jul. 30, 2021
(86) PCT No.: PCT/EP2021/071363

§ 371 Date: Feb. 3, 2023

(30) Foreign Application Priority Data
Aug. 5, 2020 (GB) 2012143.0

(51) Int. Cl. C07D 231/16; C07D 231/12; A01P 13/00; C07D 403/04; C07D 401/14; A01N 43/56; C07D 495/10

(52) U.S. Cl. CPC C07D 231/16; A01N 43/56; A01P 13/00; C07D 231/12; C07D 401/14; C07D 403/04; C07D 495/10

(57) ABSTRACT

The present invention relates novel di- and tri-substituted pyrazolo-lactam/thiolactam-carboxamide compounds, methods for their production, and intermediates for use in such methods. The invention further extends to herbicidal compositions comprising such derivatives, as well as to the use of such compounds and compositions in controlling undesirable plant growth: in particular, the use in controlling weeds in crops of useful plants.

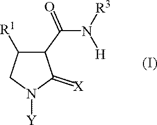

The present invention relates novel di- and tri-substituted pyrazolo-lactam/thiolactam-carboxamide derivatives, methods for their production, and intermediates for use in such methods. The invention further extends to herbicidal compositions comprising such derivatives, as well as to the use of such compounds and compositions in controlling undesirable plant growth: in particular, the use in controlling weeds in crops of useful plants.

Herbicidal pyrrolidinone derivatives are described in WO2015/084796.

The present invention is based on the finding that di- and tri-substituted pyrazolo-lactam-carboxamide derivatives of formula (I) as defined infra, exhibit surprisingly good herbicidal activity.

Thus, in a first aspect there is provided a compound of formula (I)

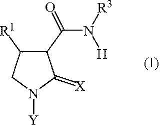

wherein;
X is O or S; Y is H, methyl, or methoxy; R¹ is 1-difluoromethyl-pyrazol-3-yl or 1-difluoromethyl-pyrazol-4-yl ring, substituted on one or both free ring carbon atom(s) by R², each R² is independently halogen, C₁—C₃fluoroalkyl, C₁—C₃haloalkoxy, C₁—C₃alkoxy, or C₁—C₃alkyl;
R³ is a phenyl, pyridinyl, or thienyl ring system, optionally substituted by 1, 2, or 3 R⁴ substituents; and each R⁴ is independently halogen, C₁—C₆alkyl, C₁—C₆haloalkyl, C₁—C₆alkoxy, C₁—C₆haloalkoxy, cyano, nitro, C₁—C₆alkylthio, C₁—C₆alkylsulfinyl, or C₁—C₆alkylsulfonyl.

According to a second aspect of the invention, there is provided an agrochemical composition comprising a compound of formula (I) and an agrochemically-acceptable diluent or carrier. Such an agricultural composition may further comprise at least one additional active ingredient.

According to a third aspect, there is provided a method of controlling unwanted plant growth, comprising applying a compound of formula (I) as defined herein, or a herbicidal composition as defined herein, to the unwanted plants or to the locus thereof.

According to a fourth aspect, there is provided the use of a compound of formula (I) as a herbicide.

Further aspects include methods of making a compound of formula (I) as described herein, as well as intermediates for use in such methods.

As used herein, the term “C₁₋₆alkyl” refers to a straight or branched hydrocarbon chain radical consisting solely of carbon and hydrogen atoms, containing no unsaturation, having from one to six carbon atoms, and which is attached to the rest of the molecule by a single bond. The term “C₁₋₄alkyl” is to be construed accordingly. Examples of C₁₋₆alkyl include, but are not limited to, methyl, ethyl, n-propyl, n-butyl, n-pentyl, n-hexyl and the isomers thereof, for example, isopropyl, iso-butyl, sec-butyl, tert-butyl or iso-amyl. A “C₁₋₄alkylene” group refers to the corresponding definition of C₁₋₄alkyl, except that such radical is attached to the rest of the molecule by two single bonds. The term “C₁₋₂alkylene” is to be construed accordingly. Examples of C₁₋₄alkylene, include, but are not limited to, —CH₂—, —CH₂CH₂— and —(CH₂)₃—.

As used herein, the term “halogen” refers to fluorine (fluoro), chlorine (chloro), bromine (bromo) or iodine (iodo). The same correspondingly applies to halogen in the context of other definitions, such as haloalkyl or haloalkoxy.

Haloalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

As used herein, the term “hydroxyl” or “hydroxy” means an —OH group.

As used herein, the term “C₁₋₆alkoxy” refers to a radical of the formula —ORₐ wherein Rₐ is a C₁₋₆alkyl radical as generally defined above. C₁₋₄alkoxy is to be construed accordingly. Examples of C₁₋₆alkoxy include, but are not limited to, methoxy, ethoxy, propoxy, iso-propoxy, and tert-butoxy. It should also be appreciated that two alkoxy substituents may be present on the same carbon atom.

As used herein, the term “C₁₋₆alkylsulfonyl” refers to a radical of the formula —S(O)₂Rₐ wherein Rₐ is a C₁₋₆alkyl radical as generally defined above. The term “C₁₋₄alkylsulfonyl” is to be construed accordingly. Preferred alkylsulfonyl radicals include methylsulfonyl, ethylsulfonyl, propylsulfonyl, isopropylsulfonyl, n-butylsulfonyl, isobutylsulfonyl, sec-butylsulfonyl or tert-butylsulfonyl. Methylsulfonyl or ethylsulfonyl are particularly preferred.

As used herein, the term “C₁₋₆alkylsulfinyl” refers to a radical of the formula —S(O)Rₐ wherein Rₐ is a C₁₋₆alkyl radical as generally defined above. The term “C₁₋₄alkylsulfinyl” is to be construed accordingly. Preferred alkylsufinyl radicals are methylsulfinyl, ethylsulfinyl, propylsulfinyl, isopropylsulfinyl, n-butylsulfinyl, isobutylsulfinyl, sec-butylsulfinyl and tert-butylsulfinyl. Methylsulfinyl and ethylsulfinyl are particularly preferred.

As used herein, the term “C₁₋₆alkylthio” refers to a radical of the formula —SRₐ wherein Rₐ is a C₁₋₆alkyl radical as generally defined above. The term “C₁₋₄alkylthio” is to be construed accordingly. Preferred alkythio examples include methylthio, ethylthio, propylthio, isopropylthio, n-butylthio, isobutylthio, sec-butylthio or tert-butylthio. Methylthio and ethylthio are particularly preferred.

As used herein, the term “cyano” means a —CN group.

As used herein, nitro means an —NO₂ group.

The compounds of formula (I) may exist as different geometric isomers, or in different tautomeric forms. This invention covers the use of all such isomers and tautomers (including lactam-lactim tautomers and keto-enol tautomers), and mixtures thereof in all proportions, as well as isotopic forms such as deuterated compounds. They may contain one or more asymmetric centers and may thus give rise to optical isomers and diastereomers. While shown for formula (I) without respect to stereochemistry, the present invention includes the use of all such optical isomers and diastereomers as well as the racemic and resolved, enantiomerically pure R and S stereoisomers and other mixtures of the R and S stereoisomers and agrochemically acceptable salts thereof. It is recognized certain optical isomers or diastereomers may have favorable properties over the other. Thus, when disclosing and claiming the invention, when a racemic mixture is disclosed, it is clearly contemplated that each optical isomer, including diastereomer, substantially free of any other, is disclosed and claimed as well.

In each case, the compounds of formula (I) according to the invention are in free form, in oxidized form as an N-oxide, in covalently hydrated form, or in salt form, e.g., an agronomically usable or agrochemically acceptable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

As stated above, the present invention also includes agronomically acceptable salts that the compounds of formula (I) may form with amines (for example ammonia, dimethylamine and triethylamine), alkali metal and alkaline earth metal bases or quaternary ammonium bases. Among the alkali metal and alkaline earth metal hydroxides, oxides, alkoxides and hydrogen carbonates and carbonates used as salt formers, emphasis is to be given to the hydroxides, alkoxides, oxides and carbonates of lithium, sodium, potassium, magnesium and calcium, but especially those of sodium, magnesium and calcium. The corresponding trimethylsulfonium salt may also be used. The compounds of formula (I) according to the invention also include hydrates which may be formed during the salt formation.

Preferred values of R¹, R², R³, R⁴, X, and Y are as set out below, and a compound of formula (I) according to the invention may comprise any combination of said values. The skilled man will appreciate that values for any specified set of embodiments may combined with values for any other set of embodiments where such combinations are not mutually exclusive.

One of the key features of compounds of formula (I) as defined herein, is that (a) the pyrazole moiety is either di-substituted, or tri-substituted, and (b) one of said substituents is borne on a ring nitrogen atom, and said substituent is difluoromethyl (—CF₂H). Such compounds have a far superior herbicidal activity in comparison to similar compounds bearing an unsubstituted pyrazole moiety, or a mono-substituted pyrazole moiety at this position, for example as described in WO2015/084796. Thus, R¹ is defined herein as a 1-difluoromethyl-pyrazol-3-yl or 1-difluoromethyl-pyrazol-4-yl ring, substituted on one or both free ring carbon atom(s) by R². It is clear to the skilled man from this description that the pyrazole moiety is thus carbon linked to the rest of the molecule.

Preferably R¹ is selected from the group consisting of R¹-1, R¹-2, R¹-3, and R¹-4,

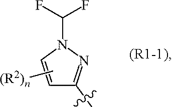

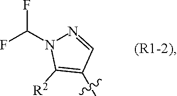

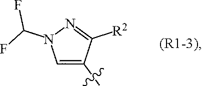

and

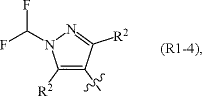

in which R² is as defined herein, n is an integer of 1 or 2, and the jagged line denotes the point of attachment to the rest of the molecule.

Preferably when R¹ is R¹-1, and n is 1, R¹ is borne by the ring carbon atom adjacent to the substituted ring nitrogen atom. R¹-1 may thus be described by the following structure

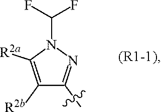

wherein R^{2a} is halogen, C₁—C₃fluoroalkyl, C₁—C₃haloalkoxy, C₁—C₃alkoxy, or C₁—C₃alkyl; R^{2b} is hydrogen, halogen, C₁—C₃fluoroalkyl, C₁—C₃haloalkoxy, C₁—C₃alkoxy, or C₁—C₃alkyl; and the jagged line denotes the point of attachment to the rest of the molecule.

In one set of preferred embodiments, R¹ is R¹-1.

R² as defined herein is halogen, C₁—C₃fluoroalkyl, C₁—C₃haloalkoxy, C₁—C₃alkoxy, or C₁—C₃alkyl. Preferably R² is halogen, C₁—C₃ alkyl or C₁—C₃fluoroalkyl. More preferably R² is fluoro, chloro, bromo, methyl, ethyl, fluoromethyl, difluoromethyl, trifluoromethyl, or difluoroethyl. More preferably still R² is fluoro, chloro, bromo, methyl or ethyl.

Where R¹ is R¹-1, R^{2a} is preferably C₁—C₃ alkyl, more preferably methyl or ethyl.

In one set of embodiments where R¹ is R¹-1, R^{2a} is preferably C₁—C₃ alkyl and R^{2b} is either hydrogen or halogen.

As stated herein, X can either be O or S. In one set of embodiments X is O. In a further set of embodiments X is S.

In all embodiment Y may be hydrogen, methyl or methoxy. However, it is preferred that Y is hydrogen or methyl. Methyl is particularly preferred.

R³ is defined herein as a phenyl, pyridinyl, or thienyl ring system, optionally substituted by 1, 2, or 3 R⁴ substituents. Preferably R³ is selected from the group consisting of R³-1, R³-2, R³-3, R³-4, R³-5, and R³-6

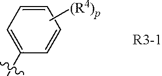

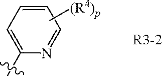

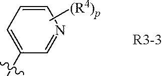

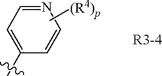

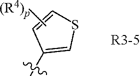

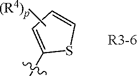

wherein p is an integer of 0, 1, 2, or 3, R⁴is as defined herein, and the jagged line represents the point of attachment to the rest of the molecule.

More preferably R³ is an optionally substituted phenyl or pyridinyl ring system, and in particular is R³-1 or R³-3.

It is preferred that p is an integer of 1 or 2.

Each R⁴ may be independently selected from the group consisting of halogen, C₁—C₆alkyl, C₁—C₆haloalkyl, C₁—C₆alkoxy, C₁—C₆haloalkoxy, cyano, nitro, C₁—C₆alkylthio, C₁—C₆alkylsulfinyl, and C₁—C₆alkylsulfonyl. Preferably each R4 is independently selected from the group consisting of halogen, C₁—C₆alkyl, C₁—C₆haloalkyl, and C₁—C₆alkoxy. More preferably each R⁴ is independently fluoro, chloro, bromo, C₁—C₃alkyl, C₁—C₃haloalkyl, or C₁—C₃alkxoy. More preferably still each R⁴ is independently fluoro, chloro, methyl, ethyl, C₁fluoroalkyl, or methoxy. In one set of embodiments each R⁴ is independently fluoro, methyl or ethyl.

In one set of embodiments R³ is selected from the group consisting of

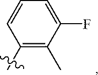

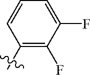

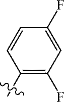

and

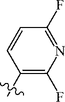

wherein the jagged line denotes the point of attachment to the rest of the molecule).

Particularly preferred compounds of formula (I) are shown below in Table 1, where they are referred to as compounds of formula (T-1). Compounds of formula (T-1) are compounds of formula (I) wherein R¹ is R¹-1 and thus has the structure

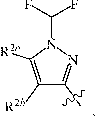

, wherein R^{2a} and R^{2b} are as defined in the table, and the jagged line denotes the point of attachment to the rest of the molecule, X, Y and R³ are as defined in the table.

Table 1 This table describes 96 specific compounds of formula (T-1)

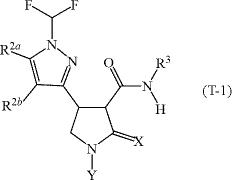

These are compounds of formula (I) wherein R^{2a}, R^{2b}, X, Y and R³ are as defined in the table below. The jagged line in R³ denotes the point of attachment to the carboxamide nitrogen.

| Compound No | R^{2a} | R^{2b} | X | Y | R³ |
|---|---|---|---|---|---|
| 1 | CH₃ | H | O | H | 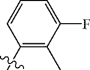 |
| 2 | C₂H₅ | H | O | H | 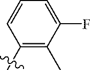 |
| 3 | CH₃ | Br | O | H | 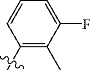 |
| 4 | C₂H₅ | Br | O | H | 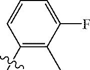 |
| 5 | CH₃ | H | S | H | 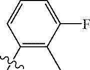 |
| 6 | C₂H₅ | H | S | H | 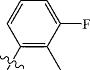 |
| 7 | CH₃ | Br | S | H | 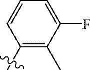 |
| 8 | C₂H₅ | Br | S | H | 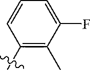 |
| 9 | CH₃ | H | O | CH₃ | 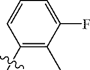 |
| 10 | C₂H₅ | H | O | CH₃ | 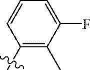 |
| 11 | CH₃ | Br | O | CH₃ | 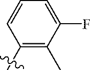 |
| 12 | C₂H₅ | Br | O | CH₃ | 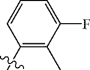 |
| 13 | CH₃ | H | S | CH₃ | 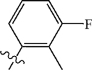 |
| 14 | C₂H₅ | H | S | CH₃ | 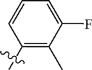 |
| 15 | CH₃ | Br | S | CH₃ | 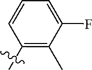 |
| 16 | C₂H₅ | Br | S | CH₃ | 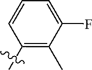 |
| 17 | CH₃ | H | O | OCH₃ | 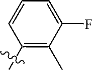 |
| 18 | C₂H₅ | H | O | OCH₃ | 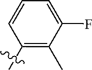 |
| 19 | CH₃ | Br | O | OCH₃ | 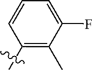 |
| 20 | C₂H₅ | Br | O | OCH₃ | 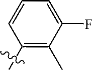 |
| 21 | CH₃ | H | S | OCH₃ | 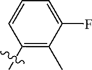 |
| 22 | C₂H₅ | H | S | OCH₃ | 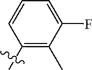 |
| 23 | CH₃ | Br | S | OCH₃ | 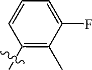 |
| 24 | C₂H₅ | Br | S | OCH₃ | 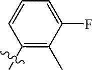 |
| 25 | CH₃ | H | O | H | 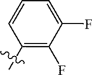 |
| 26 | C₂H₅ | H | O | H | 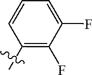 |
| 27 | CH₃ | Br | O | H | 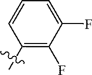 |
| 28 | C₂H₅ | Br | O | H | 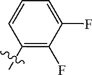 |
| 29 | CH₃ | H | S | H | 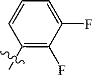 |
| 30 | C₂H₅ | H | S | H | 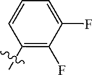 |
| 31 | CH₃ | Br | S | H | 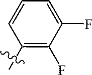 |
| 32 | C₂H₅ | Br | S | H | 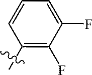 |
| 33 | CH₃ | H | O | CH₃ | 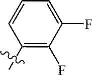 |
| 34 | C₂H₅ | H | O | CH₃ | 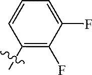 |
| 35 | CH₃ | Br | O | CH₃ | 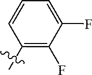 |
| 36 | C₂H₅ | Br | O | CH₃ | 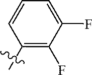 |
| 37 | CH₃ | H | S | CH₃ | 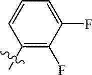 |
| 38 | C₂H₅ | H | S | CH₃ | 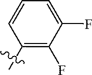 |
| 39 | CH₃ | Br | S | CH₃ | 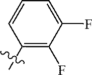 |
| 40 | C₂H₅ | Br | S | CH₃ | 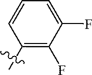 |
| 41 | CH₃ | H | O | OCH₃ | 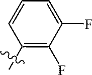 |
| 42 | C₂H₅ | H | O | OCH₃ | 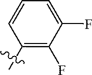 |
| 43 | CH₃ | Br | O | OCH₃ | 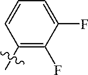 |
| 44 | C₂H₅ | Br | O | OCH₃ | 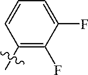 |
| 45 | CH₃ | H | S | OCH₃ | 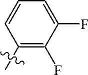 |
| 46 | C₂H₅ | H | S | OCH₃ | 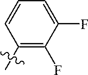 |
| 47 | CH₃ | Br | S | OCH₃ | 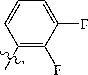 |
| 48 | C₂H₅ | Br | S | OCH₃ | 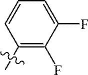 |
| 49 | CH₃ | H | O | H | 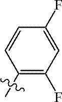 |
| 50 | C₂H₅ | H | O | H | 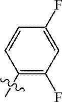 |
| 51 | CH₃ | Br | O | H | 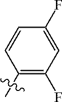 |
| 52 | C₂H₅ | Br | O | H | 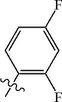 |
| 53 | CH₃ | H | S | H | 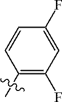 |
| 54 | C₂H₅ | H | S | H | 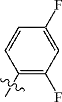 |
| 55 | CH₃ | Br | S | H | 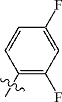 |
| 56 | C₂H₅ | Br | S | H | 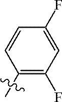 |
| 57 | CH₃ | H | O | CH₃ | 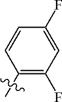 |
| 58 | C₂H₅ | H | O | CH₃ | 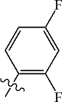 |
| 59 | CH₃ | Br | O | CH₃ | 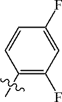 |
| 60 | C₂H₅ | Br | O | CH₃ | 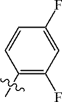 |
| 61 | CH₃ | H | S | CH₃ | 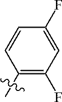 |
| 62 | C₂H₅ | H | S | CH₃ | 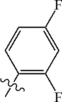 |
| 63 | CH₃ | Br | S | CH₃ | 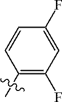 |
| 64 | C₂H₅ | Br | S | CH₃ | 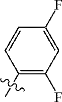 |
| 65 | CH₃ | H | O | OCH₃ | 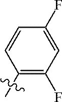 |
| 66 | C₂H₅ | H | O | OCH₃ | 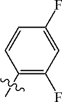 |
| 67 | CH₃ | Br | O | OCH₃ | 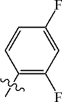 |
| 68 | C₂H₅ | Br | O | OCH₃ | 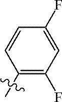 |
| 69 | CH₃ | H | S | OCH₃ | 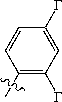 |
| 70 | C₂H₅ | H | S | OCH₃ | 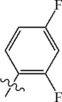 |
| 71 | CH₃ | Br | S | OCH₃ | 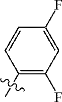 |
| 72 | C₂H₅ | Br | S | OCH₃ | 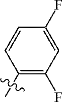 |
| 73 | CH₃ | H | O | H | 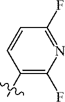 |
| 74 | C₂H₅ | H | O | H | 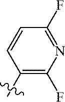 |
| 75 | CH₃ | Br | O | H | 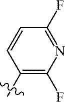 |
| 76 | C₂H₅ | Br | O | H | 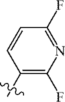 |
| 77 | CH₃ | H | S | H | 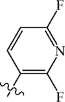 |
| 78 | C₂H₅ | H | S | H | 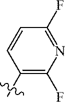 |
| 79 | CH₃ | Br | S | H | 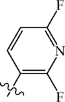 |
| 80 | C₂H₅ | Br | S | H | 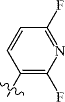 |
| 81 | CH₃ | H | O | CH₃ | 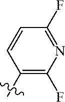 |
| 82 | C₂H₅ | H | O | CH₃ | 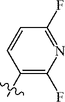 |
| 83 | CH₃ | Br | O | CH₃ | 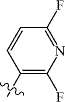 |
| 84 | C₂H₅ | Br | O | CH₃ | 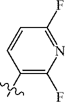 |
| 85 | CH₃ | H | S | CH₃ | 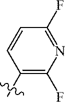 |
| 86 | C₂H₅ | H | S | CH₃ | 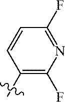 |
| 87 | CH₃ | Br | S | CH₃ | 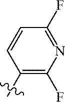 |
| 88 | C₂H₅ | Br | S | CH₃ | 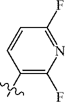 |
| 89 | CH₃ | H | O | OCH₃ | 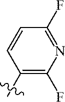 |
| 90 | C₂H₅ | H | O | OCH₃ | 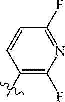 |
| 91 | CH₃ | Br | O | OCH₃ | 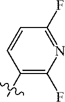 |
| 92 | C₂H₅ | Br | O | OCH₃ | 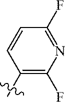 |
| 93 | CH₃ | H | S | OCH₃ | 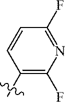 |
| 94 | C₂H₅ | H | S | OCH₃ | 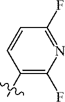 |
| 95 | CH₃ | Br | S | OCH₃ | 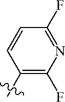 |
| 96 | C₂H₅ | Br | S | OCH₃ | 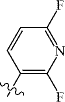 |

More preferred compounds of formula (I) are those referred to infra in the Examples.

As stated hereinbefore, a compound of formula (I) may be made and used in the form of a racemic mixture. However, enantiomers with the following stereochemistry are particularly preferred:

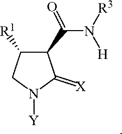

Compounds of formula (I), may be synthesised using the methods described in WO2015/084796.

The compounds of the invention, in which the substituents X, Y, R¹, R², R^{2a}, R^{2b}, R³, R⁴, n and p are as defined hereinbefore unless explicitly stated otherwise may also be synthesised from suitable halogenated pyrazoles of formula (A) according to the following general reaction scheme. The starting materials used for the preparation of compounds of formula (I) may be purchased from usual commercial suppliers or may be prepared by known methods. The starting materials as well as the intermediates may be purified before use in the next step by state-of-the-art methodologies such as chromatography, crystallization, distillation and filtration.

### Reaction Scheme 1

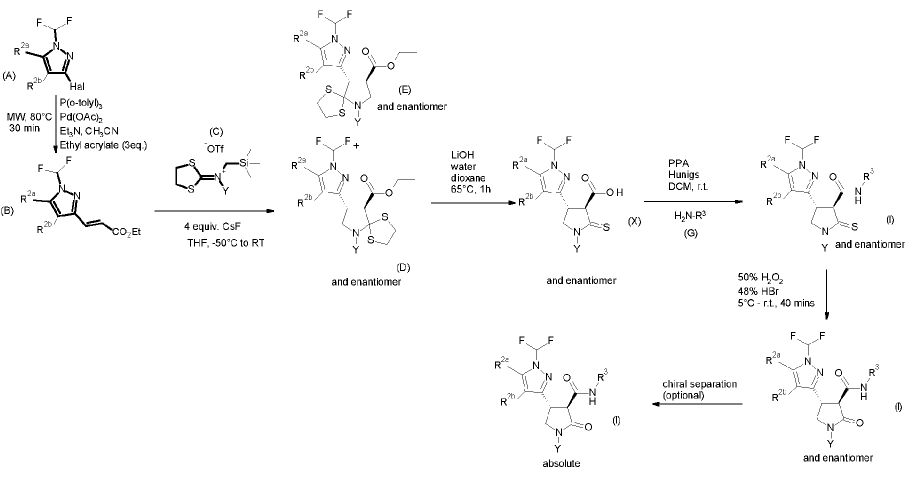 As shown in Reaction scheme 1 above, the desired halogenated pyrazole (in this case a compound of formula (A) wherein R¹ is R¹-1 R^{2a} and R^{2b} are as defined hereinbefore, and Hal is halogen) is reacted with ethyl acrylate, under palladium catalysis, to afford the substituted vinyl pyrazole of formula (B). The substituted vinyl pyrazole of formula (B) undergoes a cycloaddition with dithiolane-isocyanate imminium methylide (I) affording a mixture of pyrrolidine cycloadducts [(D), (E) and enanantiomers thereof]. These cycloadducts can be separated by chromatography. The desired pyrrolidine cycloadduct (D) is reacted with a hydroxide base, in a water/ether mixed solvent system to afford the 3-carboxyl substituted thiolactam of formula (X). The 3-carboxyl substituted thiolactam of formula (X) is coupled with an aniline of formula (G) to afford the desired amide of formula (I) using standard amide coupling conditions, such as propanephosphonic acid anhydride in a suitable solvent, such as dichloromethane, with a suitable base, such as N,N-Diisopropylethylamine. The thiolactam of formula (I) may subsequently undergo oxidative hydrolysis, with hydrogen peroxide solution and a suitable acid to afford compounds of formula (I) that are lactam derivatives. Single enantiomers can be prepared by chiral separation.

As an alternative route to lactam compounds of formula (I) (i.e. compounds of formula (I) wherein X is O), the above process may be followed as far as the production of the 3-carboxyl substituted thiolactam of formula (X). The compound of formula (X) may then undergo oxidative hydrolysis with hydrogen peroxide solution and a suitable acid, such as hydrobromic or hydrochloric acid, to afford a compound of formula (J) wherein Y is methyl as shown in Reaction scheme 2 below. The compound of formula (J) wherein Y is H or methoxy may be synthesized using the methods described in WO2015/084796. The compound of formula (J) may then be with reacted with an aniline of formula (G) using propanephosphonic acid anhydride in a suitable solvent, such as dichloromethane, with a suitable base, such as N,N- Diisopropylethylamine as shown in Reaction scheme 3 below.

### Reaction Scheme 2

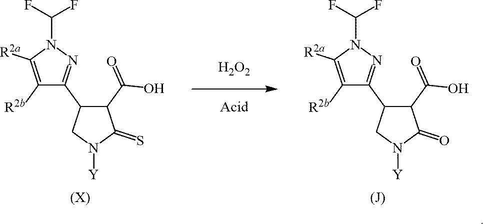

### Reaction Scheme 3

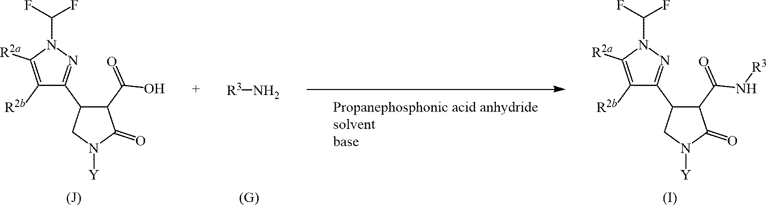

Salt (C) can be prepared as described in Tetrahedron Lett. 1995, 36, 9409.

1-(Difluromethyl) -3-iodo-pyrazoles of formula (A), in which R is methyl or ethyl and which may be employed in Reaction scheme 1 above, may be synthesised as shown in Reaction scheme 4 below.

### Reaction Scheme 4

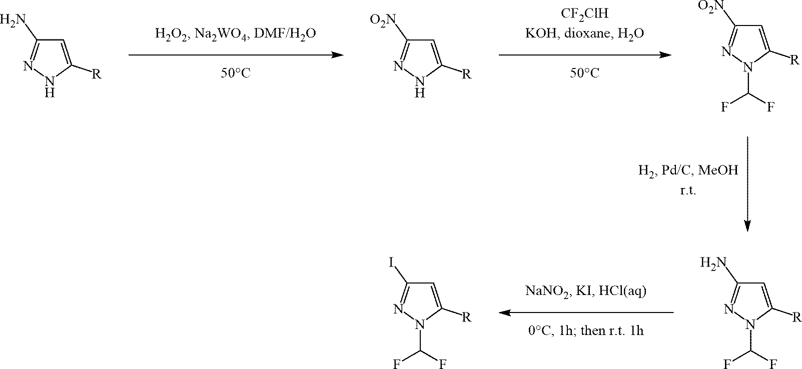

The desired 3-nitro-1H-pyrazol-5-yl can be derived via an oxidation from the corresponding 3-amino-1H-pyrazol-5-yl, with a suitable oxidant, such as hydrogen peroxide and sodium tungstate. The 3-nitro-1H-pyrazol-5-yl is then reacted with chlorodifluoromethane, with a hydroxide base in a dioxane/water mixed solvent system. The resulting 1-(difluoromethyl)-3-nitro-pyrazole is hydrogenated, with palladium on carbon and an atmosphere of hydrogen in an alcoholic solvent, such as methanol to afford the 1-(difluoromethyl)pyrazol-3-amine. The amine can be converted to the desired iodide under standard diazotisation conditions, such as sodium nitrite, with a suitable acid, such as hydrochloric acid and using potassium iodide as the iodide source.

Such processes and the intermediate compounds of formula (A), (B), (D), (E), (X) and (J) form yet further aspects of the invention.

Thus, in a further aspect there is provided a process for the production of a compound of formula (I) as defined herein and wherein X is S, said process comprising:
(i) reacting a compound of formula (A) with ethyl acrylate, under palladium catalysis to give a compound of formula (B)

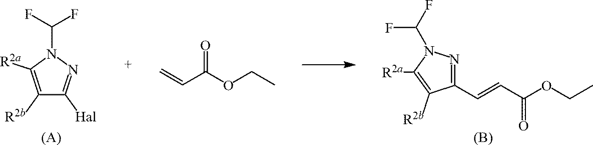

wherein
   R^{2a} halogen, C₁—C₃fluoroalkyl, C₁—C₃haloalkoxy, C₁—C₃alkoxy, or C₁—C₃alkyl;
   R^{2b} is hydrogen, halogen, C₁—C₃fluoroalkyl, C₁—C₃haloalkoxy, C₁—C₃alkoxy, or C₁—C₃alkyl; and, Hal is halogen;
(ii) reacting the compound of formula (B) from step (i) with a compound of formula (C), wherein Y is methyl in a cycloaddition reaction to yield a mixture of compounds of formula (D) and (E)

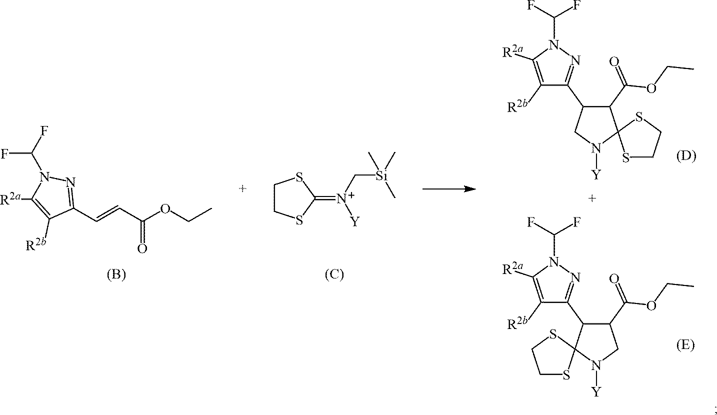

(iii) reacting the compound of formula (D) with a hydroxide base in a water/ether mixed solvent system to give the compound of formula (X)

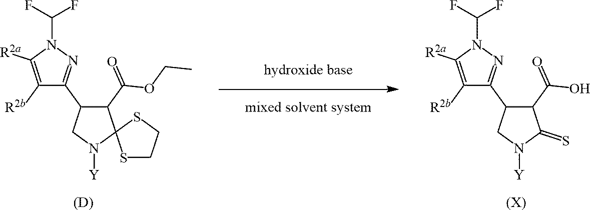

wherein R^{2a}, R^{2b}, and Y are as defined in steps (i) and (ii) above;
(iv) reacting the compound of formula (X) from step (iii) with an aniline of formula (G) using propanephosphonic acid anhydride in a suitable solvent, with a suitable base,

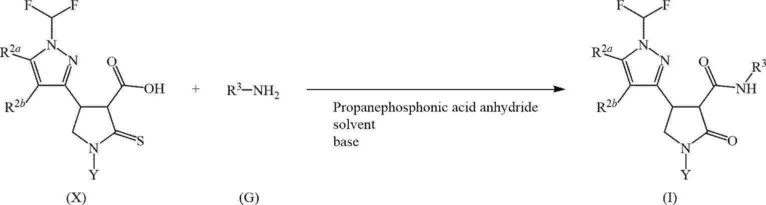

to afford the compound of formula (I), wherein R³ is a phenyl, pyridinyl, or thienyl ring system, optionally substituted by 1, 2, or 3 R⁴ substituents, and each R⁴ is independently halogen, C₁—C₆alkyl, C₁—C₆haloalkyl, C₁—C₆alkoxy, C₁—C₆haloalkoxy, cyano, nitro, C₁—C₆alkylthio, C₁—C₆alkylsulfinyl, or C₁—C₆alkylsulfonyl.

In a further aspect there is provided a process for the production of a compound of formula (I) as defined herein and wherein X is O, said process comprising steps (i) to (iv) as defined supra, and further comprising

(v) oxidatively hydrolysing the compound of formula (I) from step (iv), with hydrogen peroxide solution and a suitable acid, to yield a compound of formula (I) wherein X is O.

Since the order of the steps of oxidative hydrolysis and aniline coupling can be carried out in a different order as outline above in Reaction schemes 2 and 3, the invention provides a further process for the production of a compound of formula (I) as defined herein and wherein X is O, said process comprising steps (i) to (iii) as defined supra, and further comprising
(iv) oxidatively hydrolysing the compound of formula (X) from step (iii), with hydrogen peroxide solution and a suitable acid, to yield a compound of formula (J)

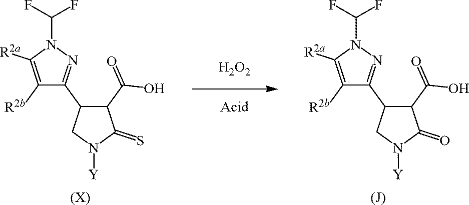

wherein
   R^{2a} halogen, C₁—C₃fluoroalkyl, C₁—C₃haloalkoxy, C₁—C₃alkoxy, or C₁—C₃alkyl;
   R^{2b} is hydrogen, halogen, C₁—C₃fluoroalkyl, C₁—C₃haloalkoxy, C₁—C₃alkoxy, or C₁—C₃alkyl; and Y is methyl; and
(v) reacting the compound of formula (J) from step (iv) with an aniline of formula (G) using propanephosphonic acid anhydride in a suitable solvent, with a suitable base,

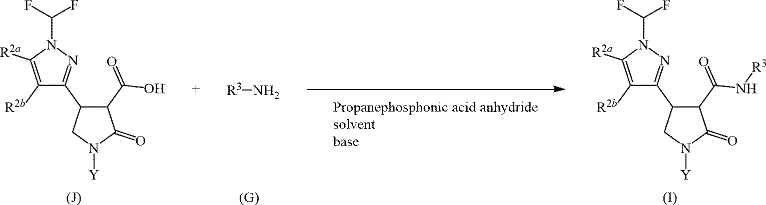

to afford the compound of formula (I), wherein R³ is a phenyl, pyridinyl, or thienyl ring system, optionally substituted by 1, 2, or 3 R⁴substituents, and each R⁴ is independently halogen, C₁—C₆alkyl, C₁—C₆haloalkyl, C₁—C₆alkoxy, C₁—C₆haloalkoxy, cyano, nitro, C₁—C₆alkylthio, C₁—C₆alkylsulfinyl, or C₁—C₆alkylsulfonyl.

As stated above, the intermediates generated in the above processes are also novel and form yet further aspects of the invention. The invention thus also provides (i) a compound of formula (B)

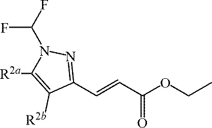

(B), wherein, R^{2a} halogen, C₁—C₃fluoroalkyl, C₁—C₃haloalkoxy, C₁—C₃alkoxy, or C₁—C₃alkyl; R^{2b} is hydrogen, halogen, C₁—C₃fluoroalkyl, C₁—C₃haloalkoxy, C₁—C₃alkoxy, or C₁—C₃alkyl;
(ii) a compound of formula (D)

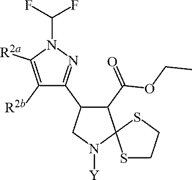

(D), wherein,R^{2a} halogen, C₁—C₃fluoroalkyl, C₁—C₃haloalkoxy, C₁—C₃alkoxy, or C₁—C₃alkyl; R^{2b} is hydrogen, halogen, C₁—C₃fluoroalkyl, C₁—C₃haloalkoxy, C₁—C₃alkoxy, or C₁—C₃alkyl; and Y is methyl;
(iii) a compound of formula (E)

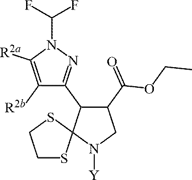

(E), wherein: R^{2a} halogen, C₁—C₃fluoroalkyl, C₁—C₃haloalkoxy, C₁—C₃alkoxy, or C₁—C₃alkyl; R^{2b} is hydrogen, halogen, C₁—C₃fluoroalkyl, C₁—C₃haloalkoxy, C₁—C₃alkoxy, or C₁—C₃alkyl; and Y is methyl,
(iv) a compound of formula (J)

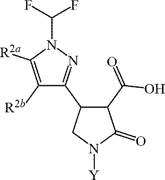

(J),wherein: R^{2a} halogen, C₁—C₃fluoroalkyl, C₁—C₃haloalkoxy, C₁—C₃alkoxy, or C₁—C₃alkyl; R^{2b} is hydrogen, halogen, C₁—C₃fluoroalkyl, C₁—C₃haloalkoxy, C₁—C₃alkoxy, or C₁—C₃alkyl; and Y is methyl;
(v) a compound of formula (X)

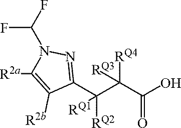

(X) wherein R^{2a} halogen, C₁—C₃fluoroalkyl, C₁—C₃haloalkoxy, C₁—C₃alkoxy, or C₁—C₃alkyl; R^{2b} is hydrogen, halogen, C₁—C₃fluoroalkyl, C₁—C₃haloalkoxy, C₁—C₃alkoxy, or C₁—C₃alkyl; R^{Q1} and R^{Q4} are each hydrogen; and R^{Q2} and R^{Q3} together with the carbon atoms to which they are joined form ring Q, which is an optionally substituted 5-membered thio-lactam ring.

Preferably in compounds of formula (X) ring Q is Q1 or Q2

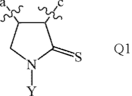

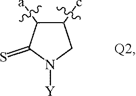

wherein Y is methyl ‘a’ denotes the point of attachment to the pyrazole moiety, and ‘c’ denotes the point of attachment to the carboxylate moiety.

Also provided by the invention is a compound of formula (A)

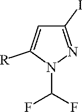

(A) wherein R is methyl or ethyl.

In yet a further aspect the invention provides the use of compounds of formula (A), (B), (D), (E), (J) and (X) as described herein, in the manufacture of a herbicide.

The compounds of formula (I) according to the invention may be used in unmodified form or, preferably, together with the adjuvants conventionally employed in the art of formulation to provide herbicidal compositions. The invention therefore further provides a herbicidal composition, comprising at least one compound of formula (I) and an agriculturally acceptable diluent or carrier and optionally an adjuvant. An agricultural acceptable carrier is for example a carrier that is suitable for agricultural use. Agricultural carriers are well known in the art. Similarly suitable agriculturally acceptable diluents are well known in the art.

To this end compounds of formula (I) may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an antifoam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller’s earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required Typical carriers for granular Formulations include sand, fuller’s earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular Formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, pdiethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller’s earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

The compounds of formula (I) are normally used in the form of agrochemical compositions and can be applied to the crop area or plant to be treated, simultaneously or in succession with further compounds/active ingredients. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

In particular, the compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, diquat dibromide, diuron, epyrifenacil, ethalfluralin, ethofumesate, fenoxaprop (including fenoxaprop-P-ethyl), fenoxasulfone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 3-[2-(3,4-dimethoxyphenyl)-6-methyl-3-oxo-pyridazine-4-carbonyl]bicyclo[3.2.1]octane-2,4-dione, 2-[2-(3,4-dimethoxyphenyl)-6-methyl-3-oxo-pyridazine-4-carbonyl]-5-methyl-cyclohexane-1,3-dione, 2-[2-(3,4-dimethoxyphenyl)-6-methyl-3-oxo-pyridazine-4-carbonyl]cyclohexane-1,3-dione, 2-[2-(3,4-dimethoxyphenyl)-6-methyl-3-oxo-pyridazine-4-carbonyl]-5,5-dimethyl-cyclohexane-1,3-dione, 6-[2-(3,4-dimethoxyphenyl)-6-methyl-3-oxo-pyridazine-4-carbonyl]-2,2,4,4-tetramethyl-cyclohexane-1,3,5-trione, 2-[2-(3,4-dimethoxyphenyl)-6-methyl-3-oxo-pyridazine-4-carbonyl]-5-ethyl-cyclohexane-1,3-dione, 2-[2-(3,4-dimethoxyphenyl)-6-methyl-3-oxo-pyridazine-4-carbonyl]-4,4,6,6-tetramethyl-cyclohexane-1,3-dione, 2-[6-cyclopropyl-2-(3,4-dimethoxyphenyl)-3-oxo-pyridazine-4-carbonyl]-5-methylcyclohexane-1,3-dione, 3-[6-cyclopropyl-2-(3,4-dimethoxyphenyl)-3-oxo-pyridazine-4-carbonyl]bicyclo[3.2.1]octane-2,4-dione, 2-[6-cyclopropyl-2-(3,4-dimethoxyphenyl)-3-oxo-pyridazine-4-carbonyl]-5,5-dimethyl-cyclohexane-1,3-dione, 6-[6-cyclopropyl-2-(3,4-dimethoxyphenyl)-3-oxo-pyridazine-4-carbonyl]-2,2,4,4-tetramethyl-cyclohexane-1,3,5-trione, 2-[6-cyclopropyl-2-(3,4-dimethoxyphenyl)-3-oxo-pyridazine-4-carbonyl]cyclohexane-1,3-dione, 4-[2-(3,4-dimethoxyphenyl)-6-methyl-3-oxo-pyridazine-4-carbonyl]-2,2,6,6-tetramethyl-tetrahydropyran-3,5-dione, 4-[6-cyclopropyl-2-(3,4-dimethoxyphenyl)-3-oxo-pyridazine-4-carbonyl]-2,2,6,6-tetramethyl-tetrahydropyran-3,5-dione, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl 2-[[3-[[3-chloro-5-fluoro-6-[3-methyl-2,6-dioxo-4-(trifluoromethyl)pyrimidin-1-yl]-2-pyridyl]oxy]acetate, 6-chloro-4-(2,7-dimethyl-1-naphthyl)-5-hydroxy-2-methyl-pyridazin-3-one, 1-[2-chloro-6-(5-chloropyrimidin-2-yl)oxy-phenyl]-4,4,4-trifluoro-butan-1-one and 5-[2-chloro-6-(5-chloropyrimidin-2-yl)oxy-phenyl]-3-(difluoromethyl)isoxazole.

The mixing partners of the compound of formula (I) may also be in the form of esters or salts, as mentioned e.g. in The Pesticide Manual, Sixteenth Edition, British Crop Protection Council, 2012.

The compound of formula (I) can also be used in mixtures with other agrochemicals such as fungicides, nematicides or insecticides, examples of which are given in The Pesticide Manual.

The mixing ratio of the compound of formula (I) to the mixing partner is preferably from 1: 100 to 1000:1.

The mixtures can advantageously be used in the above-mentioned formulations, in which case the phrase “active ingredient” relates to the respective mixture of compound of formula (I) with the mixing partner.

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of formula (I) with cyprosulfamide, isoxadifen-ethyl, cloquintocet-mexyl and/or metcamifen.

The safeners for use in combination with a compound of formula (I) may also be in the form of esters or salts, as mentioned e.g. in The Pesticide Manual, 16th Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

The compounds of formula (I) of this invention are useful as herbicides. The present invention therefore further comprises a method for controlling unwanted plants comprising applying to the said plants or a locus comprising them, an effective amount of a compound of the invention or a herbicidal composition containing said compound. The present invention still further provides method of controlling weeds at a locus said method comprising application to the locus of a weed controlling amount of a composition comprising a compound of formula (I). Moreover, the present invention may further provide a method of selectively controlling weeds at a locus comprising useful (crop) plants and weeds, wherein the method comprises application to the locus of a weed controlling amount of a composition according to the present invention. “Controlling’ means killing, reducing or retarding growth or preventing or reducing germination. Generally the plants to be controlled are unwanted plants (weeds).

“Locus” means the area in which the plants are growing or will grow.

The rates of application of compounds of formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha. The application is generally made by spraying the composition, typically by tractor mounted sprayer for large areas, but other methods such as dusting (for powders), drip or drench can also be used.

The application may be applied to the locus pre-emergence and/or postemergence of the crop plant.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes. Preferred crop plants include maize, wheat, barley and rice.

Some crop plants may be inherently tolerant to herbicidal effects of compounds of formula (I).

The term “useful plants” is to be understood as also including useful plants that have been rendered tolerant to herbicides like bromoxynil or classes of herbicides such as, for example, 4-Hydroxyphenylpyruvate dioxygenase (HPPD) inhibitors, ALS inhibitors, for example primisulfuron, prosulfuron and trifloxysulfuron, 5-enol-pyrovyl-shikimate-3-phosphate-synthase (EPSPS) inhibitors, glutamine synthetase (GS) inhibitors or protoporphyrinogen-oxidase (PPO) inhibitors as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex 10 and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGardÒ (maize variety that expresses a CrylA(b) toxin); YieldGard RootwormÒ (maize variety that expresses a CryIIIB(b1) toxin); YieldGard PlusÒ (maize variety that expresses a CryIA(b) and a CryIIIB(b1) toxin); StarlinkÒ (maize variety that expresses a Cry9(c) toxin); Herculex IÒ (maize variety that expresses a CrylF(a2) toxin and the enzyme phosphinothricine N-acetyltransferase (PAT) to achieve tolerance to the herbicide glufosinate ammonium); NuCOTN 33BÒ (cotton variety that expresses a CryIA(c) toxin); Bollgard lÒ (cotton variety that expresses a CryIA(c) toxin); Bollgard II® (cotton variety that expresses a CryIA(c) and a CryllA(b) toxin); VIPCOTÒ (cotton variety that expresses a VIP toxin); NewLeafÒ (potato variety that expresses a CryIIIA toxin); NatureGardÒ Agrisure® GT Advantage (GA21 glyphosate-tolerant trait), Agrisure® CB Advantage (Bt11 corn borer (CB) trait), Agrisure® RW (corn rootworm trait) and ProtectaÒ.

Crops/useful plants are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention. The Examples which follow serve to illustrate, but do not limit, the invention.

### EXAMPLES

### Formulation Examples

| Wettable powders | a) | b) | c) |
|---|---|---|---|
| active ingredients | 25% | 50% | 75% |
| sodium lignosulfonate | 5% | 5% | - |
| sodium lauryl sulfate | 3% | - | 5% |
| sodium diisobutylnaphthalenesulfonate | - | 6% | 10% |
| phenol polyethylene glycol ether | - | 2% | - |
| (7-8 mol of ethylene oxide) | - | - | - |
| highly dispersed silicic acid | 5% | 10% | 10% |
| Kaolin | 62% | 27% | - |

The combination is thoroughly mixed with the adjuvants and the mixture is thoroughly ground in a suitable mill, affording wettable powders that can be diluted with water to give suspensions of the desired concentration.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | - | - |
| Kaolin | - | 94 % | - |
| mineral filler | - | | 96% |

Ready-for-use dusts are obtained by mixing the combination with the carrier and grinding the mixture in a suitable mill.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

| Suspension concentrate | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75 % emulsion in water) | 1% |
| Water | 32% |

The finely ground combination is intimately mixed with the adjuvants, giving a suspension concentrate from which suspensions of any desired dilution can be obtained by dilution with water.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### Preparative Examples

### General Experimental

Chiral HPLC was recorded on the columns below with the solvents and gradients stated. Column:
Regis Whelk O1 (s,s) 4.6 × 100 mm, 3.5 µm
Chiralpak IC 4.6 × 100 mm, 3.0 µm

Solvents:
A: iso-Hexane + 0.1% glacial Acetic Acid (v/v)
B: Ethanol + 0.1% glacial Acetic Acid (v/v)

Gradient

| Time (mins): | Flow (mL/min): | %A: | %B: |
|---|---|---|---|
| 0.0 | 1.0 | 85 | 15 |
| 1.0 | 1.0 | 85 | 15 |
| 7.0 | 1.0 | 50 | 50 |
| 15.0 | 1.0 | 40 | 60 |

### Example P1 Synthesis of 1-(difluoromethyl)-3-iodo-5-methyl-pyrazole - Compound (A1)

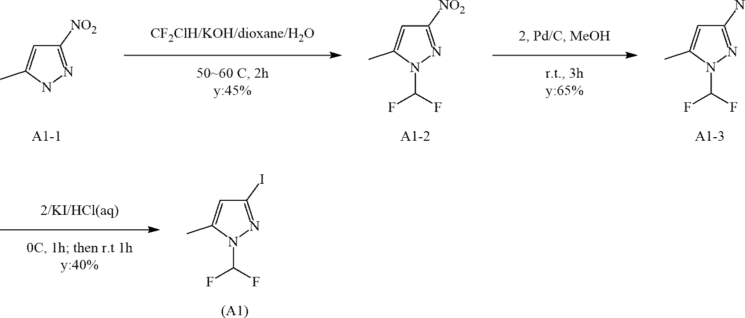

### Step 1: Synthesis of Compound (A1- 2)

Chlorodifluoromethane was bubbled into a mixture of compound (A1-1) (6.3 g, 50 mmol) and KOH (8.5 g, 150 mmol) in 50 ml of dioxane and 30 ml of water at 50° C. for about 2 h. After cooling to room temperature, the resulting mixture was poured into water, and the mixture was extracted three times with diethyl ether. The combined organic phases were dried over anhydrous sodium sulfate and concentrated under vacuum. The residue was purified by column chromatography on silica gel (petroleum ether / ethyl acetate = 10:1) to give the compound (A1-2) (3.9 g, 45% yield) as a light yellow solid.

¹H NMR (400 Mz, CDCl₃): δ 2.53 (s, 3H), 6.76 (s, 1H), 7.22 (t, J=58.2 Hz, 1H); ¹⁹F NMR (282 MHz, CDCl₃): δ -95.2(d, J=58.3 Hz, 2F).

### Step 2: Synthesis of Compound (A1-3)

A mixture of compound A1-2 (1.77 g, 20 mmol) and 10% of Pd/C (500 mg) in 30 ml MeOH was hydrogenated at room temperature for about 3 h. The reaction mixture was filtered and the solvent was removed under vacuum. The residue was purified by column chromatography on silica gel (petroleum ether / ethyl acetate = 5:1) to give the compound (A1-3) (0.95 g, 65% yield) as a yellow oil.

¹H NMR (400 Mz, CDCl₃): δ 2.33 (s, 3H), 3.96 (br s, 2H), 5.56 (s, 1H), 6.92 (t, J=59.7 Hz, 1H); ¹⁹F NMR (282 MHz, CDCl₃): δ -92.5(d, J=59.8 Hz, 2F).

### Step 3: Synthesis of Compound (A1)

Sodium nitrite (0.83 g, 12 mmol) dissolved in 5 ml of water was added dropwise to a mixture of compound 3 (1.47 g, 10 mmol) and 4N hydrochloric acid (50 ml) at 0° C. After the addition was complete, the resulting mixture was stirred for one hour at the same temperature. Then, a solution of potassium iodide (3.32 g, 20 mmol) in 10 ml of water was added dropwise to the mixture. The reaction mixture was warmed to room temperature and stirred for another one hour. The mixture was extracted three times with ethyl acetate, and the combined organics were washed with a saturated aqueous solution of sodium thiosulfate, then dried over anhydrous sodium sulfate, filtered and concentrated in vacuo. The residue was purified by column chromatography on silica gel (petroleum ether) to give the compound(A1) (1 g, 40% yield) as a light yellow solid.

¹H NMR (400 Mz, CDCl₃): δ 2.43 (s, 3H), 6.29 (s, 1H), 7.16 (t, J=58.8 Hz, 1H); ¹⁹F NMR (282 MHz, CDCl₃): δ -94.8(d, J=58.6 Hz, 2F).

### Example P2 Synthesis of 1-(difluoromethyl)-3-iodo-5-ethyl-pyrazole - Compound (A2)

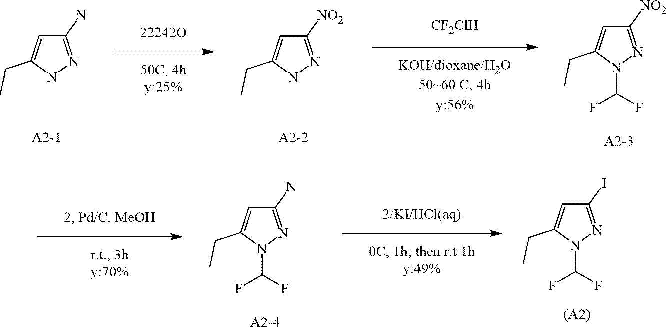

### Step 1: Synthesis of Compound (A2-2)

30% H₂O₂ (10 g, 90 mmol) was added dropwise to a mixture of compound (A2-1) (3.33 g, 30 mmol) and Na₂WO₄ (1.98 g, 6 mmol) in 30 ml of DMF and 10 ml of water at 50° C. After the addition, the mixture was stirred at 50° C. for 4 hour. After cooling to room temperature, the resulting mixture was poured into water, and the mixture was extracted three times with diethyl ether. The combined organic phases were dried over anhydrous sodium sulfate and concentrated under vacuum. The residue was purified by column chromatography on silica gel (petroleum ether / ethyl acetate = 5:1) to give the compound (A2-2) (105 g, 25% yield) as a yellow solid. ¹H NMR (400 Mz, DMSO-d₆): δ 1.18 (t, J=6.8 Hz, 3H), 2.63 (q, J=7.2 Hz, 2H), 6.79 (s, 1H), 13.67 (br s, 1H).

### Step 2: Synthesis of Compound (A2-3)

Chlorodifluoromethane was bubbled into a mixture of compound (A2-2) (1.41 g, 10 mmol) and KOH (1.71 g, 30 mmol) in 20 ml of dioxane and 10 ml of water at 50° C. for about 4 h. After cooling to room temperature, the resulting mixture was poured into water, and the mixture was extracted three times with diethyl ether. The combined organic phases were dried over anhydrous sodium sulfate and concentrated under vacuum. The residue was purified by column chromatography on silica gel (petroleum ether / ethyl acetate = 10:1) to give the compound (A2-3) (1.06 g, 56% yield) as a light yellow solid.

¹H NMR (400 Mz, CDCl₃): δ1.35 (t, J=7.6 Hz, 3H), 2.92 (q, J=7.6 Hz, 2H), 6.81 (s, 1H), 7.22 (t, J=57.6 Hz, 1H); ¹⁹F NMR (282 MHz, CDCl₃): δ -94.5(d, J=57.8 Hz, 2F).

### Step 3: Synthesis of Compound (A2-4)

A mixture of compound (A2-3) (1.91 g, 10 mmol) and 10% of Pd/C (500 mg) in 30 ml MeOH was hydrogenated at room temperature for about 3 h. The reaction mixture was filtered and the solvent was removed under vacuum give a crude compound (A2-4) (1.12 g, 70% yield) as a yellow oil, which was used for the next step without further purification.

### Step 4: Synthesis of Compound (A2)

Sodium nitrite (0.58 g, 8.4 mmol) dissolved in 5 ml of water was added dropwise to a mixture of compound (A2-4) (1.12 g, 7 mmol) and 4N hydrochloric acid (40 ml) at 0° C. After the addition was complete, the resulting mixture was stirred for one hour at the same temperature. Then, a solution of potassium iodide (2.32 g, 14 mmol) in 10 ml of water was added dropwise to the mixture. The reaction mixture was warmed to room temperature and stirred for another one hour. The mixture was extracted three times with ethyl acetate, and the combined organics were washed with a saturated aqueous solution of sodium thiosulfate, then dried over anhydrous sodium sulfate, filtered and concentrated in vacuo. The residue was purified by column chromatography on silica gel (petroleum ether) to give the compound (A2) (0.93 g, 49% yield) as a light yellow solid.

¹H NMR (400 Mz, CDCl₃).. δ1.28 (t, J=7.6 Hz, 3H), 2.83 (q, J=7.6 Hz, 2H), 6.33 (s, 1H), 7.16 (t, J=59.2 Hz, 1H); ¹⁹F NMR (282 MHz, CDCl₃): δ -94.1 (d, J=57.8 Hz, 2F).

### Example p3: Synthesis of 4-[1-(difluoromethyl)-5-methyl-pyrazol-3-yl]-n-(2,3-difluorophenyl)-1-methyl-2-thioxo-pyrrolidine-3-carboxamide (Compound No. 37) and 4-[1-(difluoromethyl)-5-methyl-pyrazol-3-yl]-n-(2,3-difluorophenyl)-1-methyl-2-oxo-pyrrolidine-3-carboxamide (Compound No33)

### Step 1: Synthesis of Ethyl (E)-3-[1-(difluoromethyl)-5-methyl-pyrazol-3-yl]prop-2-enoate

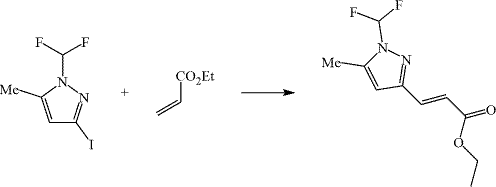

1-(Difluoromethyl)-3-iodo-5-methyl-pyrazole (0.20 g, 0.78 mmol) was dissolved in acetonitrile (2.7 mL) in a microwave vial, and ethyl acrylate (0.25 mL, 2.3 mmol) was added followed by triethylamine (0.11 mL, 0.78 mmol), tri-ortho-tolylphoshine (0.024 g, 0.078 mmol) and palladium acetate (0.017 g, 0.078 mmol). The vial was flushed with nitrogen, sealed and heated at 100oC under microwave irradiation for 30 minutes.The reaction mixture was filtered, rinsing through with small portions of EtOAc, and the combined filtrate and washings were concentrated to remove the bulk of solvent. The crude product was diluted with water (10 mL) and extracted with EtOAc (3 x 15 mL). The organic extracts were then combined, washed with water (2 x 10 mL), passed through a phase separation cartridge and the collected organics concentrated giving a light brown oil. Purification by chromatography with an EtOAc/iso-hexane gradient elution, afforded the desired product, ethyl (E)-3-[1-(difluoromethyl)-5-methyl-pyrazol-3-yl]prop-2-enoate, as light brown liquid (144 mg).

1H NMR: (400 MHz, CDCl3) δ = 7.56 (d, J = 16.1 Hz, 1H), 7.33 - 7.04 (t, 1H), 6.40 (d, 1H), 6.36 (s, 1H), 4.26 (q, J = 7.1 Hz, 2H), 2.47 (s, 3H), 1.33 (t, J = 7.1 Hz, 3H)

### Step 2: Synthesis of Ethyl-8-[1-(difluoromethyl)-5-methyl-pyrazol-3-yl]-6-methyl-1,4-dithia-6-azaspiro[4.4]nonane-9-carboxylate

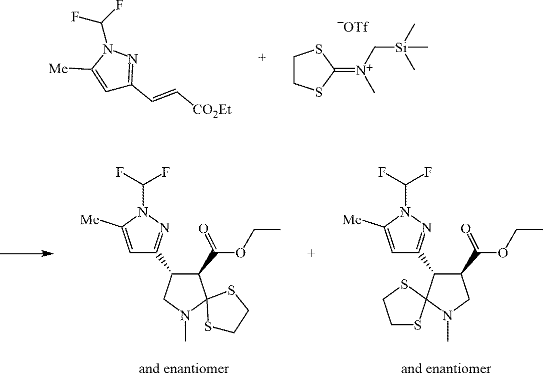

To a suspension of cesium fluoride (0.370 g, 2.43 mmol) in tetrahydrofuran (1.8 mL) stirred at -50° C., under a nitrogen atmosphere was added a solution of ethyl (E)-3-[1-(difluoromethyl)-5-methyl-pyrazol-3-yl]prop-2-enoate (0.140 g, 0.608 mmol) and 1,3-dithiolan-2-ylidene-methyl-(trimethylsilylmethyl)ammonium;trifluoromethanesulfonate (0.338 g, 0.912 mmol) in tetrahydrofuran (1.82 mL) drop-wise over 10 minutes, whilst keeping the reaction temperature below -45° C. The resulting pale amber suspension was allowed to warm to room temperature gradually. After 16 hours the reaction mixture was diluted with dichloromethane and filtered. Purification by chromatography with an EtOAc/iso-hexane gradient elution afforded ethyl-8-[1-(difluoromethyl)-5-methyl-pyrazol-3-yl]-6-methyl-1,4-dithia-6-azaspiro[4.4]nonane-9-carboxylate as a yellow oil, (94 mg)

1H NMR: (400 MHz, CDCl3)δ = 7.27 - 6.97 (t, 1H), 6.02 (s, 1H), 4.31 - 4.17 (m, 2H), 3.87 (d, 1H), 3.78 (q, J = 8.4 Hz, 1H), 3.31 - 3.05 (m, 5H), 2.94 (dd, J = 7.5, 9.3 Hz, 1H), 2.46 (s, 3H), 2.40 (s, 3H), 1.31 (t, J = 7.2 Hz, 3H)

### Step 3: Synthesis of 4-[1-(difluoromethyl)-5-methyl-pyrazol-3-yl]-1-methyl-2-thioxo-pyrrolidine-3-carboxylic Acid

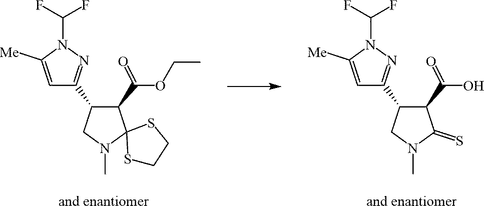

To a solution of ethyl-8-[1-(difluoromethyl)-5-methyl-pyrazol-3-yl]-6-methyl-1,4-dithia-6-azaspiro[4.4]nonane-9-carboxylate (0.094 g, 0.25 mmol,) in 1,4-dioxane (6 mL) and water (2 mL,) was added lithium hydroxide (0.060 g, 2.5 mmol,) and the stirred mixture heated to 60° C. under a nitrogen atmosphere. After 45 mins the reaction mixture was allowed to cool and concentrated to remove the bulk of dioxane. The residual mixture was diluted with water (10 mL), acidified with dilute HCl to pH3, then extracted with DCM (3 × 8 mL). The organic extracts were combined, washed with water (5 mL), separated then passed through a phase separation cartridge. The collected organics were concentrated to give a colourless gum which crystallised to an off-white solid, 4-[1-(difluoromethyl)-5-methyl-pyrazol-3-yl]-1-methyl-2-thioxo-pyrrolidine-3-carboxylic acid (73 mg).

1H NMR: (400 MHz, CDCl3) δ = 7.24 - 6.95 (t, 1H), 6.22 (s, 1H), 4.17 - 4.02 (m, 4H), 3.33 (s, 3H), 2.43 (s, 3H)

### Step 4: Synthesis of 4-[1-(difluoromethyl)-5-methyl-pyrazol-3-yl]-N-(2,3-difluorophenyl)-1-methyl-2-thioxo-pyrrolidine-3-carboxamide (Compound No. 37)

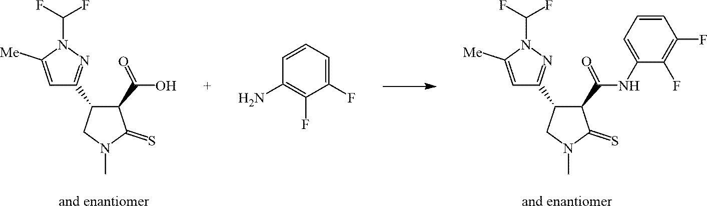

To a solution of 4-[1-(difluoromethyl)-5-methyl-pyrazol-3-yl]-1-methyl-2-thioxo-pyrrolidine-3-carboxylic acid (0.073 g, 0.25 mmol,) in dichloromethane (1.8 mL) was added 2,3-difluoroaniline (0.026 mL, 0.25 mmol). The propylphosphonic anhydride (50 mass% in ethyl acetate) (0.26 mL, 0.43 mmol,) was added, followed by N,N-diisopropylethylamine (0.13 mL, 0.76 mmol). The reaction mixture was stirred at room temperature for 2 hours then left to stand at room temp overnight.

The reaction mixture was quenched by the addition of water (2 mL), with stirring, transferred to a phase separation cartridge and the organics collected and the collected and purified by chromatography with an EtOAc/iso-hexane gradient elution to afford 4-[1-(difluoromethyl)-5-methyl-pyrazol-3-yl]-N-(2,3-difluorophenyl)-1-methyl-2-thioxo-pyrrolidine-3-carboxamide as a colourless gum (62 mg).

1H NMR: (400 MHz, CDCl3) δ = 10.21 (br s, 1H), 8.05 - 7.98 (m, 1H), 7.26 - 6.95 (t, 1H), 7.04 (ddt, J = 2.1, 5.9, 8.3 Hz, 1H), 6.95 - 6.86 (m, 1H), 6.13 (s, 1H), 4.40 - 4.33 (m, 1H), 4.19 (d, J = 6.1 Hz, 1H), 4.11 (dd, 1H), 4.02 (dd, 1H), 3.32 (s, 3H), 2.43 (d, 3H)

### Step 5: Synthesis of 4-[1-(difluoromethyl)-5-methyl-pyrazol-3-yl]-N-(2,3-difluorophenyl)-1-methyl-2-oxo-pyrrolidine-3-carboxamide (Compound No. 33)

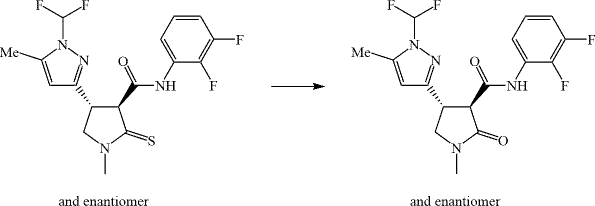 [SMILES: CCc1cc([C@H]2CN(C)C(=O)C2C(=O)Nc2ccc(F)nc2F)nn1C(F)F]

To a solution of 4-[1-(difluoromethyl)-5-methyl-pyrazol-3-yl]-N-(2,3-difluorophenyl)-1-methyl-2-thioxo-pyrrolidine-3-carboxamide (0.060 g, 0.15 mmol,) in acetonitrile (1.5 mL), stirred and cooled to around 0-5° C., in an ice bath, was added hydrogen peroxide (50%) (0.18 mL). After 5 minutes hydrobromic acid (0.018 mL, 0.15 mmol) was added and the colourless solution was stirred at 5° C. for around 1 h. The reaction mixture was quenched with sodium thiosulfate solution (2 mL) and the mixture was diluted with water (2 mL) and concentrated to remove the bulk of solvent. The residual aqueous was extracted with DCM (3 × 5 mL) and the combined organic extracts passed through a phase separation cartridge. The collected organics were concentrated to give a colourless gum.The crude product was purified by chromatography with an EtOAc/iso-hexane gradient elution, affording 4-[1-(difluoromethyl)-5-methyl-pyrazol-3-yl]-N-(2,3-difluorophenyl)-1-methyl-2-oxo-pyrrolidine-3-carboxamide as a colourless gum (50 mg).

1H NMR: (400 MHz, CDCl3) δ = 10.14 (br s, 1H), 8.08 - 8.02 (m, 1H), 7.27 - 6.98 (t, 1H), 7.02 (ddt, J = 2.1, 5.9, 8.3 Hz, 1H), 6.93 - 6.84 (m, 1H), 6.26 (s, 1H), 4.07 (q, J = 8.9 Hz, 1H), 3.76 (d, J = 9.3 Hz, 1H), 3.72 (s, 1H), 3.70 (s, 1H), 2.97 (d, 3H), 2.44 (d, 3H).

The following compounds of formula (I), shown below in Table 2, were made in an analogous manner.

Table 2 Compounds of formula (I)

| | | | |
|---|---|---|---|
| Cmpd. No. | Name | Structure | 1HNMR (CDCl₃) |
| 82 | 4-[1-(difluoromethyl)-5-ethyl-pyrazol-3-yl]-N-(2,6-difluoro-3-pyridyl)-1-methyl-2-oxo-pyrrolidine-3-carboxamide | 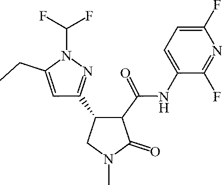 [SMILES: CCc1cc([C@H]2CN(C)C(=O)C2C(=O)Nc2cccc(F)c2C)nn1C(F)F] | δ = 10.13 (br s, 1H), 8.84 - 8.76 (m, 1H), 7.27 - 6.98 (t [large fluorine coupling], 1H), 6.80 (dd, J = 3.1, 8.6 Hz, 1H), 6.26 (s, 1H), 4.06 (q, J = 8.8 Hz, 1H), 3.79 (d, J = 9.3 Hz, 1H), 3.76 - 3.67 (m, 2H), 2.98 (d, 3H), 2.83 (q, J = 7.6 Hz, 2H), 1.31 (t, J = 7.5 Hz, 3H) |
| | | racemate | |
| 10 | 4-[1-(difluoromethyl)-5-ethyl-pyrazol-3-yl]-N-(3-fluoro-2-methylphenyl)-1-methyl-2-oxo-pyrrolidine-3-carboxamide | 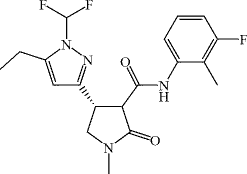 [SMILES: CCc1cc([C@H]2CN(C)C(=O)C2C(=O)Nc2ccc(F)cc2F)nn1C(F)F] | 9.78 (s, 1H), 7.87 (d, J = 8.2 Hz, 1H), 7.27 - 6.97 (t [large fluorine coupling], 1H), 7.13 (q, 1H), 6.81 (t, J = 8.7 Hz, 1H), 6.30 (s, 1H), 4.11 (q, J = 8.9 Hz, 1H), 3.79 - 3.69 (m, 3H), 2.97 (d, J = 0.7 Hz, 3H), 2.83 (q, J = 7.6 Hz, 2H), 2.27 (d, J = 1.7 Hz, 3H), 1.30 (t, J = 7.5 Hz, 3H) |
| | | Racemate | |
| 58 | 4-[1-(difluoromethyl)-5-ethyl-pyrazol-3-yl]-N-(2,4-difluorophenyl)-1-methyl-2-oxo-pyrrolidine-3-carboxamide | 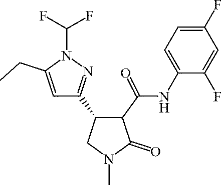 [SMILES: CCc1cc([C@H]2CN(C)C(=O)C2C(=O)Nc2cccc(F)c2F)nn1C(F)F] | 9.94 (br s, 1H), 8.23 (dt, J = 6.0, 8.9 Hz, 1H), 7.27 - 6.97 (t [large fluorine coupling], 1H), 6.90 - 6.81 (m, 2H), 6.28 (s, 1H), 4.09 (q, J = 8.9 Hz, 1H), 3.75 (d, J = 9.3 Hz, 1H), 3.72 (d, 2H), 2.97 (d, J = 0.7 Hz, 3H), 2.83 (q, J = 7.5 Hz, 2H), 1.30 (t, J = 7.5 Hz, 3H) |
| | | racemate | |
| 35 | 4-[1-(difluoromethyl)-5-ethyl-pyrazol-3-yl]-N-(2,3-difluorophenyl)-1-methyl-2-oxo-pyrrolidine-3-carboxamide | 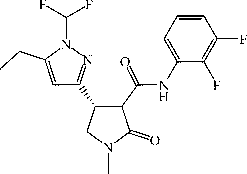 [SMILES: Cc1c(F)cccc1NC(=O)C1C(=O)N(C)C[C@@H]1c1cc(C)n(C(F)F)n1] | 10.11 (br s, 1H), 8.05 (tdd, J = 1.6, 6.6, 8.3 Hz, 1H), 7.27 - 6.98 (t [large fluorine coupling], 1H), 7.02 (ddt, J = 2.1, 5.9, 8.3 Hz, 1H), 6.88 (dddd, J = 1.6, 7.3, 8.5, 9.8 Hz, 1H), 6.28 (s, 1H), 4.09 (q, J = 8.8 Hz, 1H), 3.78 (d, J = 9.2 Hz, 1H), 3.72 (d, 2H), 2.97 (d, J = 0.7 Hz, 3H), 2.83 (q, J = 7.6 Hz, 2H), 1.30 (t, J = 7.6 Hz, 3H) |
| | | racemate | |
| 9 | 4-[1-(difluoromethyl)-5-methyl-pyrazol-3-yl]-N-(3-fluoro-2-methyl-phenyl)-1-methyl-2-oxo-pyrrolidine-3-carboxamide | 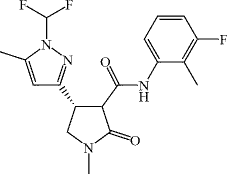 [SMILES: Cc1c(Br)c([C@H]2CN(C)C(=O)C2C(=O)Nc2ccc(F)nc2F)nn1C(F)F] | 9.79 (br s, 1H), 7.86 (d, J = 8.2 Hz, 1H), 7.27 - 6.97 (t [large fluorine coupling], 1H), 7.12 (q, 1H), 6.81 (t, J = 8.8 Hz, 1H), 6.28 (s, 1H), 4.09 (q, 1H), 3.79 -3.67 (m, 3H), 2.97 (s, 3H), 2.44 (d, 3H), 2.27 (d, J = 1.7 Hz, 3H) |
| | | racemate | |
| 83 | 4-[4-bromo-1-(difluoromethyl)-5-methyl-pyrazol-3-yl]-N-(2,6-difluoro-3-pyridyl)-1-methyl-2-oxo-pyrrolidine-3-carboxamide | 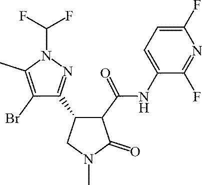 [SMILES: CCc1cc([C@H]2CN(C)C(=S)C2C(=O)Nc2ccc(F)nc2F)nn1C(F)F] | 10.03 (br s, 1H), 8.84 - 8.69 (m, 1H), 7.22 - 6.93 (t [large fluorine coupling], 1H), 6.78 (dd, J = 2.9, 8.6 Hz, 1H), 4.20 - 4.15 (m, 2H), 3.86 - 3.81 (m, 1H), 3.43 - 3.35 (m, 1H), 2.97 (s, 3H), 2.45 (s, 3H) |
| | | racemate | |
| 86 | 4-[1-(difluoromethyl)-5-ethyl-pyrazol-3-yl]-N-(2,6-difluoro-3-pyridyl)-1-methyl-2-thioxo-pyrrolidine-3-carboxamide | 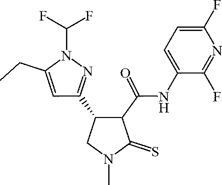 [SMILES: Cc1c(Br)c([C@H]2CN(C)C(=O)C2C(=O)Nc2ccc(F)cc2F)nn1C(F)F] | 10.31 (br s, 1H), 8.77 (dt, J = 7.2, 9.0 Hz, 1H), 7.27 - 6.96 (t [large fluorine coupling], 1H), 6.81 (dd, J = 2.9, 8.6 Hz, 1H), 6.15 (s, 1H), 4.36 (q, 1H), 4.21 (d, J = 6.5 Hz, 1H), 4.15 - 4.00 (m, 2H), 3.33 (s, 3H), 2.82 (q, J = 7.5 Hz, 2H), 1.29 (t, J = 7.5 Hz, 3H) |
| | | racemate | |
| 59 | 4-[4-bromo-1-(difluoromethyl)-5-methyl-pyrazol-3-yl]-N-(2,4-difluorophenyl)-1-methyl-2-oxo-pyrrolidine-3-carboxamide | 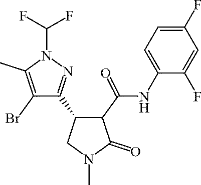 [SMILES: Cc1cc([C@H]2CN(C)C(=O)C2C(=O)Nc2ccc(F)nc2F)nn1C(F)F] | 9.84 (br s, 1H), 8.23 (dt, J = 6.0, 8.9 Hz, 1H), 7.23 - 6.92 (t [large fluorine coupling], 1H), 6.90 - 6.79 (m, 2H), 4.23 - 4.14 (m, 2H), 3.86 - 3.80 (m, 1H), 3.41 - 3.35 (m, 1H), 2.96 (s, 3H), 2.44 (s, 3H) |
| | | racemate | |
| 81 | 4-[1-(difluoromethyl)-5-methyl-pyrazol-3-yl]-N-(2,6-difluoro-3-pyridyl)-1-methyl-2-oxo-pyrrolidine-3-carboxamide | 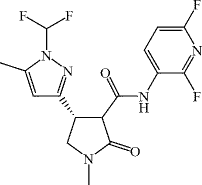 [SMILES: CCc1cc(C2CN(C)C(=S)C2C(=O)Nc2cccc(F)c2C)nn1C(F)F] | 10.14 (br s, 1H), 8.79 (dt, J = 7.2, 9.0 Hz, 1H), 7.27 - 6.98 (t [large fluorine coupling], 1H), 6.80 (dd, J = 2.9, 8.6 Hz, 1H), 6.24 (s, 1H), 4.04 (q, J = 8.8 Hz, 1H), 3.78 (d, J = 9.4 Hz, 1H), 3.75 - 3.66 (m, 2H), 2.98 (d, 3H), 2.45 (d, 3H) |
| | | racemate | |
| 14 | 4-[1-(difluoromethyl)-5-ethyl-pyrazol-3-yl]-N-(3-fluoro-2-methyl-phenyl)-1-methyl-2-thioxo-pyrrolidine-3-carboxamide | Racemate | 9.78 (br s, 1H), 7.77 (d, J = 8.2 Hz, 1H), 7.27 - 6.96 (t [large fluorine coupling], 1H), 7.14 (q, 1H), 6.84 (t, J = 8.6 Hz, 1H), 6.16 (s, 1H), 4.43 (td, J = 5.4, 8.3 Hz, 1H), 4.19 (d, J = 5.6 Hz, 1H), 4.13 (dd, 1H), 4.06 (dd, 1H), 3.33 (s, 3H), 2.82 (q, J = 7.5 Hz, 2H), 2.29 (d, J = 1.7 Hz, 3H), 1.29 (t, J = 7.5 Hz, 3H) |
| | | 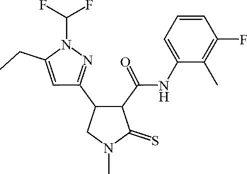 [SMILES: CCc1cc(C2CN(C)C(=S)C2C(=O)Nc2ccc(F)cc2F)nn1C(F)F] | |
| 62 | 4-[1-(difluoromethyl)-5-ethyl-pyrazol-3-yl]-N-(2,4-difluorophenyl)-1-methyl-2-thioxo-pyrrolidine-3-carboxamide | 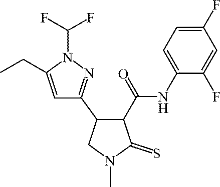 [SMILES: CCc1cc(C2CN(C)C(=S)C2C(=O)Nc2cccc(F)c2F)nn1C(F)F] | 10.02 (br s, 1H), 8.20 (dd, J = 5.9, 8.9 Hz, 1H), 7.26 -6.95 (t [large fluorine coupling], 1H), 6.91 - 6.82 (m, 2H), 6.15 (s, 1H), 4.41 - 4.36 (m, 1H), 4.18 (d, J = 6.2 Hz, 1H), 4.12 (dd, 1H), 4.04 (dd, 1H), 3.33 (s, 3H), 2.82 (q, J = 7.5 Hz, 2H), 1.29 (t, 3H) |
| | | racemate | |
| 38 | 4-[1-(difluoromethyl)-5-ethyl-pyrazol-3-yl]-N-(2,3-difluorophenyl)-1-methyl-2-thioxo-pyrrolidine-3-carboxamide | 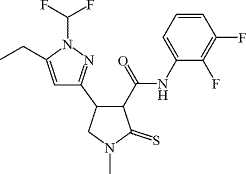 [SMILES: Cc1cc(C2CN(C)C(=O)C2C(=O)Nc2ccc(F)cc2F)nn1C(F)F] | 10.21 (s, 1H), 8.05 -7.99 (m, 1H), 7.26 -6.96 (t [large fluorine coupling], 1H), 7.07 - 7.01 (m, 1H), 6.90 (dddd, J = 1.5, 7.3, 8.5, 9.8 Hz, 1H), 6.15 (s, 1H), 4.43 - 4.35 (m, 1H), 4.20 (d, J = 6.1 Hz, 1H), 4.16 - 4.01 (m, 2H), 3.33 (s, 3H), 2.82 (q, J = 7.6 Hz, 2H), 1.29 (t, J = 7.5 Hz, 3H) |
| | | racemate | |
| 57 | 4-[1-(difluoromethyl)-5-methyl-pyrazol-3-yl]-N-(2,4-difluorophenyl)-1-methyl-2-oxo-pyrrolidine-3-carboxamide | 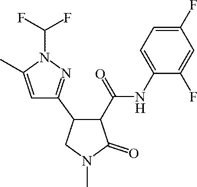 [SMILES: Cc1c(F)cccc1NC(=O)C1C(=S)N(C)CC1c1cc(C)n(C(F)F)n1] | 9.95 (br s, 1H), 8.23 (dt, J = 6.0, 8.9 Hz, 1H), 7.28 - 6.95 (t [large fluorine coupling], 1H), 6.90 - 6.80 (m, 2H), 6.25 (s, 1H), 4.06 (q, J = 8.9 Hz, 1H), 3.76 -3.68 (m, 3H), 2.97 (s, 3H), 2.44 (d, J = |
| | | racemate | 0.7 Hz, 3H) |
| 13 | 4-[1-(difluoromethyl)-5-methyl-pyrazol-3-yl]-N-(3-fluoro-2-methyl-phenyl)-1-methyl-2-thioxo-pyrrolidine-3-carboxamide | 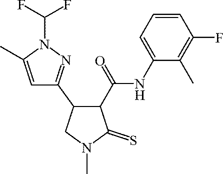 [SMILES: Cc1cc(C2CN(C)C(=S)C2C(=O)Nc2ccc(F)nc2F)nn1C(F)F] | 9.77 (br s, 1H), 7.77 (d, J = 8.2 Hz, 1H), 7.26 - 6.96 (t [large fluorine coupling], 1H), 7.13 (q, 1H), 6.84 (t, J = 8.8 Hz, 1H), 6.13 (s, 1H), 4.44 - 4.36 (m, 1H), 4.20 - 4.09 (m, 2H), 4.03 (dd, 1H), 3.33 (s, 3H), 2.43 (d, J = 0.9 Hz, 3H), 2.29 (s, 3H) |
| | | racemate | |
| 85 | 4-[1-(difluoromethyl)-5-methyl-pyrazol-3-yl]-N-(2,6-difluoro-3-pyridyl)-1-methyl-2-thioxo-pyrrolidine-3-carboxamide | 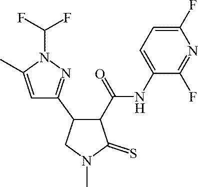 [SMILES: Cc1cc(C2CN(C)C(=S)C2C(=O)Nc2ccc(F)cc2F)nn1C(F)F] | 10.30 (br s, 1H), 8.77 (dt, J = 7.2, 9.0 Hz, 1H), 7.26 - 6.96 (t [large fluorine coupling], 1H), 6.81 (dd, J = 2.9, 8.6 Hz, 1H), 6.13 (s, 1H), 4.34 (q, 1H), 4.19 (d, J = 6.6 Hz, 1H), 4.10 (dd, 1H), 4.02 (dd, 1H), 3.33 (s, 3H), 2.43 (d, J = 0.9 Hz, 3H) |
| | | racemate | |
| 61 | 4-[1-(difluoromethyl)-5-methyl-pyrazol-3-yl]-N-(2,4-difluorophenyl)-1-methyl-2-thioxo-pyrrolidine-3-carboxamide | 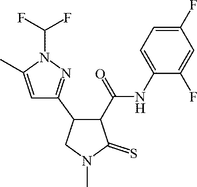 [SMILES: Cc1cc(C2CN(C)C(=O)C2C(=O)Nc2cccc(F)c2F)nn1C(F)F] | 10.01 (br s, 1H), 8.23 - 8.15 (m, 1H), 7.26 - 6.96 (t [large fluorine coupling], 1H), 6.91 - 6.82 (m, 2H), 6.12 (s, 1H), 4.37 (td, J = 5.9, 8.4 Hz, 1H), 4.19 - 4.08 (m, 2H), 4.02 (dd, 1H), 3.32 (s, 3H), 2.43 (d, 3H) |
| | | racemate | |
| 33 | 4-[1-(difluoromethyl)-5-methyl-pyrazol-3-yl]-N-(2,3-difluorophenyl)-1-methyl-2-oxo-pyrrolidine-3-carboxamide | 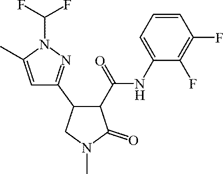 [SMILES: Cc1cc(C2CN(C)C(=S)C2C(=O)Nc2cccc(F)c2F)nn1C(F)F] | 10.14 (br s, 1H), 8.08 - 8.02 (m, 1H), 7.27 - 6.98 (t [large fluorine coupling], 1H), 7.02 (ddt, J = 2.1, 5.9, 8.3 Hz, 1H), 6.93 - 6.84 (m, 1H), 6.26 (s, 1H), 4.07 (q, J = 8.9 Hz, 1H), 3.76 (d, J = 9.3 Hz, 1H), 3.72 (s, 1H), 3.70 (s, 1H), 2.97 (d, 3H), 2.44 (d, 3H) |
| | | racemate | |
| 37 | 4-[1-(difluoromethyl)-5-methyl-pyrazol-3-yl]-N-(2,3-difluorophenyl)-1-methyl-2-thioxo-pyrrolidine-3-carboxamide | 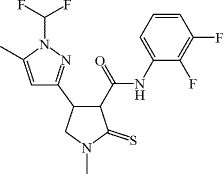 | 10.21 (br s, 1H), 8.05 - 7.98 (m, 1H), 7.26 - 6.95 (t [large fluorine coupling], 1H), 7.04 (ddt, J = 2.1, 5.9, 8.3 Hz, 1H), 6.95 - 6.86 (m, 1H), 6.13 (s, 1H), 4.40 - 4.33 (m, 1H), 4.19 (d, J = 6.1 Hz, 1H), 4.11 (dd, 1H), |
| | | racemate | 4.02 (dd, 1H), 3.32 (s, 3H), 2.43 (d, 3H) |

### Biological Examples

### Herbicidal Efficacy of Compounds of Formula (I)

Seeds of a variety of test species [Ipomoea hederacea (IPOHE); Zea mays (ZEAMX); Echinochloa crus-galli (ECHCG); Setaria faberi (SETFA); Abutilon theophrasti (ABUTH); Amaranthus retroflexus (AMARE)] were sown in standard sterilised soil in pots. After cultivation for one day (pre-emergence) or after 8 days cultivation (post-emergence) under controlled conditions in a glasshouse (at 24/16° C., day/night; 14 hours light; 65 % humidity), the plants were sprayed with an aqueous spray solution derived from the formulation of the technical active ingredient in acetone / water (50:50) solution containing 0.5% Tween 20 (polyoxyethelyene sorbitan monolaurate, CAS RN 9005-64-5), subsequently diluted in water, and sprayed to give the stated application rate.

The test plants were then grown under controlled conditions in a glasshouse (at 24/16° C., day/night; 14 hours light; 65 % humidity) and watered twice daily.

After 13 days for pre- and post-emergence, the test was evaluated visually for percentage phytotoxicity to the plant (where 5 = total damage to plant; 0 = no damage to plant). Results are shown in Tables B1 and B2.

Table B1 Application pre-emergence

| Compound Number | Rate (g/ha) | SETFA | ECHCG | ZEAMX | IPOHE | AMARE | ABUTH |
|---|---|---|---|---|---|---|---|
| 82 | 250 | 5 | 5 | 3 | 3 | 1 | 1 |
| 10 | 250 | 5 | 5 | 4 | 2 | 1 | 2 |
| 58 | 250 | 5 | 5 | 3 | 1 | 0 | 0 |
| 35 | 250 | 5 | 5 | 4 | 1 | 2 | 2 |
| 9 | 250 | 5 | 5 | 3 | 1 | 1 | 1 |
| 83 | 250 | 5 | 5 | 1 | 0 | 0 | 0 |
| 86 | 250 | 5 | 5 | 1 | 0 | 2 | 1 |
| 59 | 250 | 5 | 5 | 1 | 0 | 0 | 1 |
| 81 | 250 | 5 | 5 | 2 | 2 | 0 | 0 |
| 62 | 250 | 5 | 5 | 2 | 0 | 0 | 0 |
| 38 | 250 | 5 | 5 | 4 | 2 | 0 | 0 |
| 57 | 250 | 5 | 5 | 2 | 1 | 1 | 1 |
| 85 | 250 | 4 | 4 | 1 | 0 | 0 | 0 |
| 61 | 250 | 5 | 5 | 1 | 0 | 2 | 0 |
| 33 | 250 | 5 | 5 | 4 | 1 | 2 | 1 |

Table B2 Application post-emergence

| Compound Number | Rate (g/ha) | SETFA | ECHCG | ZEAMX | IPOHE | AMARE | ABUTH |
|---|---|---|---|---|---|---|---|
| 82 | 250 | 4 | 4 | 4 | 4 | 1 | 1 |
| 10 | 250 | 4 | 4 | 4 | 4 | 1 | 0 |
| 58 | 250 | 4 | 4 | 2 | 3 | 1 | 0 |
| 35 | 250 | 4 | 4 | 4 | 4 | 2 | 0 |
| 9 | 250 | 4 | 4 | 3 | 3 | 3 | 0 |
| 83 | 250 | 3 | 4 | 4 | 1 | 1 | 1 |
| 86 | 250 | 4 | 4 | 3 | 1 | 1 | 3 |
| 59 | 250 | 4 | 4 | 3 | 1 | 1 | 0 |
| 81 | 250 | 4 | 4 | 4 | 1 | 2 | 0 |
| 62 | 250 | 4 | 4 | 1 | 1 | 1 | 1 |
| 38 | 250 | 4 | 4 | 3 | 4 | 1 | 0 |
| 57 | 250 | 4 | 4 | 2 | 3 | 1 | 1 |
| 85 | 250 | 2 | 4 | 1 | 1 | 1 | 0 |
| 61 | 250 | 3 | 4 | 2 | 1 | 1 | 0 |
| 33 | 250 | 4 | 4 | 3 | 4 | 2 | 1 |

1. A compound of formula (I) or an N-oxide or salt thereof 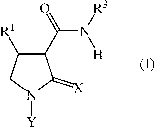
wherein;
X is O or S;
Y is H, methyl, or methoxy;
R¹ is 1-difluoromethyl-pyrazol-3-yl or 1-difluoromethyl-pyrazol-4-yl ring, substituted on one or both free ring carbon atom(s) by R²,
each R² is independently halogen, C₁-C₃fluoroalkyl, C₁-C₃haloalkoxy, C₁-C₃alkoxy, or C₁-C₃alkyl;
R³ is a phenyl, pyridinyl, or thienyl ring system, optionally substituted by 1, 2, or 3 R⁴ substituents; and
each R⁴ is independently halogen, C₁-C₆alkyl, C₁-C₆haloalkyl, C₁-C₆alkoxy, C₁-C₆haloalkoxy, cyano, nitro, C₁-C₆alkylthio, C₁-C₆alkylsulfinyl, or C₁-C₆alkylsulfonyl.

2. The compound according to claim 1, wherein R¹ is selected from the group consisting of
R¹-1, R¹-2, R¹-3, and R¹-4, 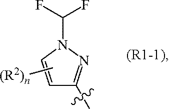 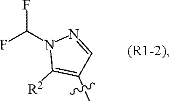 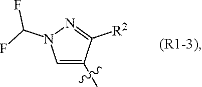
and 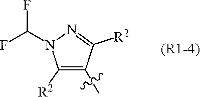
and wherein each R² is as defined in claim 1, n is an integer of 1 or 2, and the jagged line denotes the point of attachment to the rest of the molecule.

3. The compound according to claim 1, wherein Y is H or methyl.

4. The compound according to claim 1 wherein X is S.

5. The compound according to claim 1 wherein X is O.

6. The compound according to claim 1, wherein R³ is selected from the group consisting of R³-1, R³-2, R³-3, R³-4, R³-5, and R³- 6 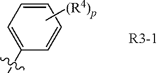 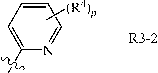 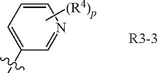 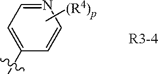 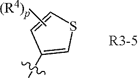 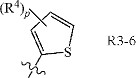
wherein p is an integer of 0, 1, 2, or 3, R⁴ is as defined in claim 1, and the jagged line represents the point of attachment to the rest of the molecule.

7. The compound according to claim 1 wherein each R⁴ is independently halogen, C₁-C₄ alkyl, C₁-C₃ haloalkyl, C₁-C₃alkoxy, or C₁-C₃haloalkoxy.

8. The compound according to claim 1, which is selected from the group of compounds shown in the table below:

| Cmpd. No. | Name | Structure |
|---|---|---|
| 9 | 4-[1-(difluoromethyl)-5-methyl-pyrazol-3-yl]-N-(3-fluoro-2-methyl-phenyl)-1-methyl-2-oxo-pyrrolidine-3-carboxamide | 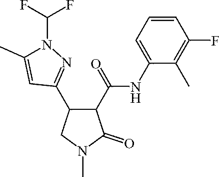 [SMILES: CCc1cc(C2CN(C)C(=O)C2C(=O)Nc2cccc(F)c2C)nn1C(F)F] |
| 10 | 4-[1-(difluoromethyl)-5-ethyl-pyrazol-3-yl]-N-(3-fluoro-2-methyl-phenyl)-1-methyl-2-oxo-pyrrolidine-3-carboxamide | 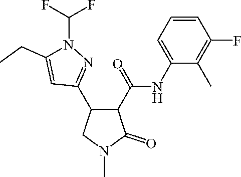 [SMILES: Cc1c(F)cccc1NC(=O)C1C(=S)N(C)CC1c1cc(C)n(C(F)F)n1] |
| 13 | 4-[1-(difluoromethyl)-5-methyl-pyrazol-3-yl]-N-(3-fluoro-2-methyl-phenyl)-1-methyl-2-thioxo-pyrrolidine-3-carboxamide | 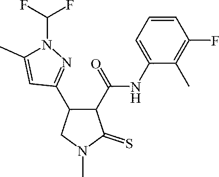 [SMILES: Cc1cc(C2CN(C)C(=S)C2C(=O)Nc2cccc(F)c2F)nn1C(F)F] |
| 14 | 4-[1-(difluoromethyl)-5-methyl-pyrazol-3-yl]-N-(2,3-difluorophenyl)-1-methyl-2-thioxo-pyrrolidine-3-carboxamide | 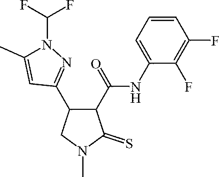 [SMILES: Cc1cc(C2CN(C)C(=O)C2C(=O)Nc2cccc(F)c2F)nn1C(F)F] |
| 33 | 4-[1-(difluoromethyl)-5-methyl-pyrazol-3-yl]-N-(2,3-difluorophenyl)-1-methyl-2-oxo-pyrrolidine-3-carboxamide | 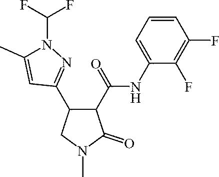 [SMILES: CCc1cc(C2CN(C)C(=O)C2C(=O)Nc2cccc(F)c2F)nn1C(F)F] |
| 35 | 4-[1-(difluoromethyl)-5-ethyl-pyrazol-3-yl]-N-(2,3-difluorophenyl)-1-methyl-2-oxo-pyrrolidine-3-carboxamide | 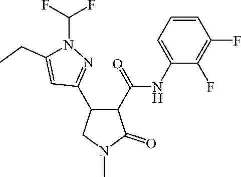 [SMILES: Cc1cc(C2CN(C)C(=S)C2C(=O)Nc2cccc(F)c2F)nn1C(F)F] |
| 37 | 4-[1-(difluoromethyl)-5-methyl-pyrazol-3-yl]-N-(2,3-difluorophenyl)-1-methyl-2-thioxo-pyrrolidine-3-carboxamide | 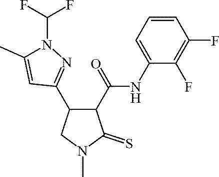 [SMILES: CCc1cc(C2CN(C)C(=S)C2C(=O)Nc2cccc(F)c2F)nn1C(F)F] |
| 38 | 4-[1-(difluoromethyl)-5-ethyl-pyrazol-3-yl]-N-(2,3-difluorophenyl)-1-methyl-2-thioxo-pyrrolidine-3-carboxamide | 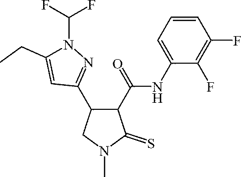 [SMILES: Cc1cc(C2CN(C)C(=O)C2C(=O)Nc2ccc(F)cc2F)nn1C(F)F] |
| 57 | 4-[1-(difluoromethyl)-5-methyl-pyrazol-3-yl]-N-(2,4-difluorophenyl)-1-methyl-2-oxo-pyrrolidine-3-carboxamide | 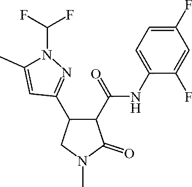 [SMILES: CCc1cc(C2CN(C)C(=O)C2C(=O)Nc2ccc(F)cc2F)nn1C(F)F] |
| 58 | 4-[1-(difluoromethyl)-5-ethyl-pyrazol-3-yl]-N-(2,4-difluorophenyl)-1-methyl-2-oxo-pyrrolidine-3-carboxamide | 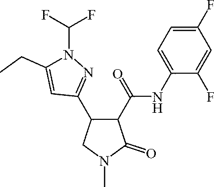 [SMILES: Cc1c(Br)c(C2CN(C)C(=O)C2C(=O)Nc2ccc(F)cc2F)nn1C(F)F] |
| 59 | 4-[4-bromo-1-(difluoromethyl)-5-methyl-pyrazol-3-yl]-N-(2,4-difluorophenyl)-1-methyl-2-oxo-pyrrolidine-3-carboxamide | 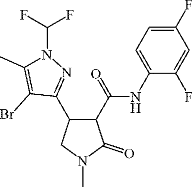 [SMILES: Cc1cc(C2CN(C)C(=S)C2C(=O)Nc2ccc(F)cc2F)nn1C(F)F] |
| 61 | 4-[1-(difluoromethyl)-5-methyl-pyrazol-3-yl]-N-(2,4-difluorophenyl)-1-methyl-2-thioxo-pyrrolidine-3-carboxamide | 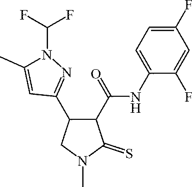 [SMILES: CCc1cc(C2CN(C)C(=S)C2C(=O)Nc2ccc(F)cc2F)nn1C(F)F] |
| 62 | 4-[1-(difluoromethyl)-5-ethyl-pyrazol-3-yl]-N-(2,4-difluorophenyl)-1-methyl-2-thioxo-pyrrolidine-3-carboxamide | 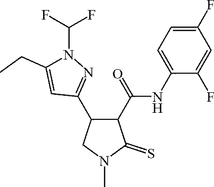 [SMILES: Cc1cc(C2CN(C)C(=O)C2C(=O)Nc2ccc(F)nc2F)nn1C(F)F] |
| 81 | 4-[1-(difluoromethyl)-5-methyl-pyrazol-3-yl]-N-(2,6-difluoro-3-pyridyl)-1-methyl-2-oxo-pyrrolidine-3-carboxamide | 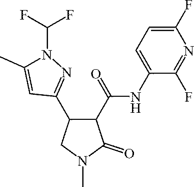 [SMILES: CCc1cc(C2CN(C)C(=O)C2C(=O)Nc2ccc(F)nc2F)nn1C(F)F] |
| 82 | 4-[1-(difluoromethyl)-5-ethyl-pyrazol-3-yl]-N-(2,6-difluoro-3-pyridyl)-1-methyl-2-oxo-pyrrolidine-3-carboxamide | 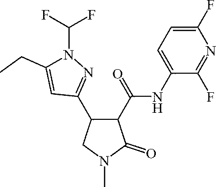 [SMILES: Cc1c(Br)c(C2CN(C)C(=O)C2C(=O)Nc2ccc(F)nc2F)nn1C(F)F] |
| 83 | 4-[4-bromo-1-(difluoromethyl)-5-methyl-pyrazol-3-yl]-N-(2,6-difluoro-3-pyridyl)-1-methyl-2-oxo-pyrrolidine-3-carboxamide | 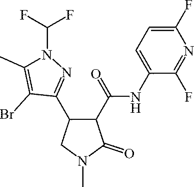 [SMILES: Cc1cc(C2CN(C)C(=S)C2C(=O)Nc2ccc(F)nc2F)nn1C(F)F] |
| 85 | 4-[1-(difluoromethyl)-5-methyl-pyrazol-3-yl]-N-(2,6-difluoro-3-pyridyl)-1-methyl-2-thioxo-pyrrolidine-3-carboxamide | 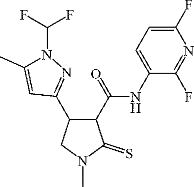 [SMILES: CCc1cc(C2CN(C)C(=S)C2C(=O)Nc2ccc(F)nc2F)nn1C(F)F] |
| 86 | 4-[1-(difluoromethyl)-5-ethyl-pyrazol-3-yl]-N-(2,6-difluoro-3-pyridyl)-1-methyl-2-thioxo-pyrrolidine-3-carboxamide | 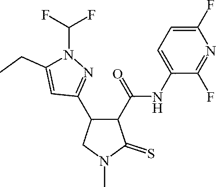 |

.

9. An agrochemical composition comprising a herbicidally effective amount of a compound of formula (I) as defined in claim 1 and an agrochemically-acceptable diluent or carrier.

10. The agrochemical composition according to claim 9, comprising at least one further pesticide.

11. A method of controlling unwanted plant growth, comprising applying a compound of formula (I) as defined in claim 1, to the unwanted plants or to the locus thereof.

12. Use of a compound of formula (I) as defined in claim 1, as a herbicide.

13. A process for the production of a compound of formula (I) as defined in claim 4, said process comprising:
(i) reacting a compound of formula (A) with ethyl acrylate, under palladium catalysis to give a compound of formula (B) 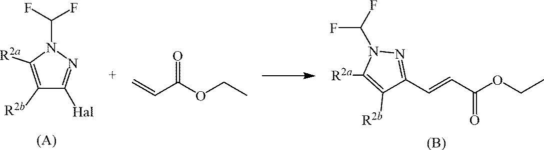
wherein
R2^{a} halogen, C₁—C₃fluoroalky1, C₁—C₃haloalkoxy, C₁—C₃alkoxy, or C₁—C₃alkyl;
R^{2b} is hydrogen, halogen, C₁—C₃fluoroalky1, C₁—C₃haloalkoxy, C₁—C₃alkoxy, or C₁-
C₃alkyl; and,
Hal is halogen;
(ii) reacting the compound of formula (B) from step (i) with a compound of formula (C), wherein Y is methyl.
in a cycloaddition reaction to yield a mixture of compounds of formula (D) and (E) 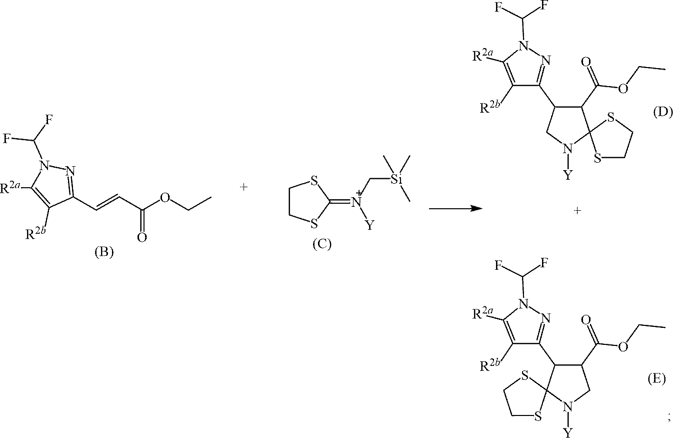
(iii) reacting the compound of formula (D) with a hydroxide base in a water/ether mixed solvent system to give the compound of formula (X) 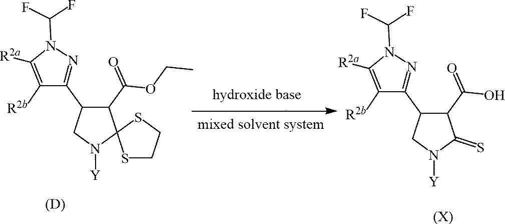
wherein R
^{2a}, R^{2b}, and Y are as defined in steps (i) and (ii) above;
(iv) reacting the compound of formula (X) from step (iii) with an aniline of formula (G)
using propanephosphonic acid anhydride in a suitable solvent, with a suitable base, 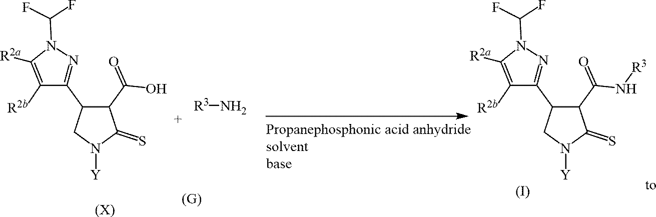
afford the compound of formula (I), wherein R
³ is a phenyl, pyridinyl, or thienyl ring system, optionally substituted by 1, 2, or 3 R⁴ substituents, and each R⁴ is independently halogen, C₁—C₆alkyl, C₁—C₆haloalkyl, C₁—C₆alkoxy, C₁—C₆haloalkoxy, cyano, nitro, C₁—C₆alkylthio, C₁—C₆alkylsulfinyl, or C₁—C₆alkylsulfonyl.

14. A process for the production of a compound of formula (I) as defined in claim 5 and further comprising
(v) oxidatively hydrolysing the compound of formula (I) from step (iv), with hydrogen peroxide solution and a suitable acid, to yield a compound of formula (I) as defined in claim 5.

15. A process for the production of a compound of formula (I) as defined in claim 5 and further comprising
(iv) oxidatively hydrolysing the compound of formula (X) from step (iii), with hydrogen peroxide solution and a suitable acid, to yield a compound of formula 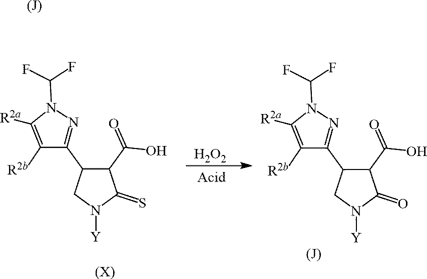
wherein
R2^{a} halogen, C₁—C₃fluoroalky1, C₁—C₃haloalkoxy, C₁—C₃alkoxy, or C₁—C₃alkyl;
R^{2b} is hydrogen, halogen, C₁—C₃fluoroalky1, C₁—C₃haloalkoxy, C₁—C₃alkoxy, or C₁—
C₃alkyl; and
Y is methyl;
(v) reacting the compound of formula (J) from step (iv) with an aniline of formula (G)
using propanephosphonic acid anhydride in a suitable solvent, with a suitable base, 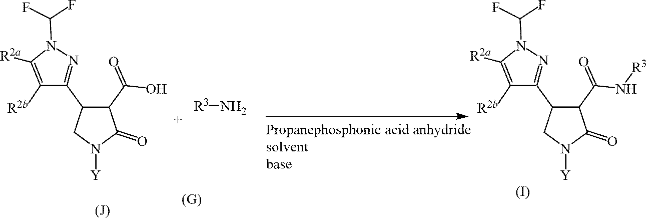
to afford the compound of formula (I), wherein R
³ is a phenyl, pyridinyl, or thienyl ring system, optionally substituted by 1, 2, or 3 R⁴ substituents, and each R⁴ is independently halogen, C₁—C₆alkyl, C₁—C₆haloalkyl, C₁—C₆alkoxy, C₁—C₆haloalkoxy, cyano, nitro, C₁—C₆alkylthio, C₁—C₆alkylsulfinyl, or C₁—C₆alkylsulfonyl.

16. A compound of formula (A) 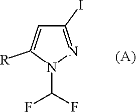
wherein R is methyl or ethyl.

17. A compound of formula (B) 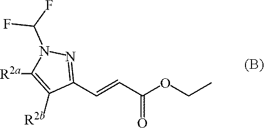
wherein,
R2^{a} halogen, C₁—C₃fluoroalky1, C₁—C₃haloalkoxy, C₁—C₃alkoxy, or C₁—C₃alkyl;
R^{2b} is hydrogen, halogen, C₁—C₃fluoroalky1, C₁—C₃haloalkoxy, C₁—C₃alkoxy, or C₁—
C₃alkyl; and
Y is methyl.

18. A compound of formula (D) 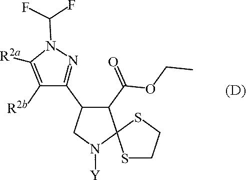
wherein,
R2^{a} halogen, C₁—C₃fluoroalky1, C₁—C₃haloalkoxy, C₁—C₃alkoxy, or C₁—C₃alkyl;
R^{2b} is hydrogen, halogen, C₁—C₃fluoroalky1, C₁—C₃haloalkoxy, C₁—C₃alkoxy, or C₁—
C₃alkyl; and
Y is methyl.

19. A compound of formula (E) 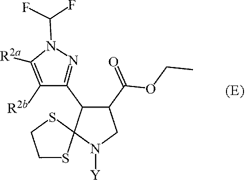
wherein:
R2^{a} halogen, C₁—C₃fluoroalky1, C₁—C₃haloalkoxy, C₁—C₃alkoxy, or C₁—C₃alkyl;
R^{2b} is hydrogen, halogen, C₁—C₃fluoroalkyl, C₁—C₃haloalkoxy, C₁—C₃alkoxy, or C₁—
C₃alkyl; and
Y is methyl.

20. A compound of formula (J) 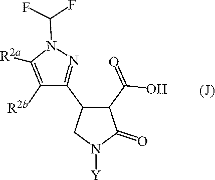
wherein:
R2^{a} halogen, C₁—C₃fluoroalkyl, C₁—C₃haloalkoxy, C₁—C₃alkoxy, or C₁—C₃alkyl;
R^{2b} is hydrogen, halogen, C₁—C₃fluoroalkyl, C₁—C₃haloalkoxy, C₁—C₃alkoxy, or C₁—
C₃alkyl; and
Y is H, methyl, or methoxy.

21. A compound of formula (X) 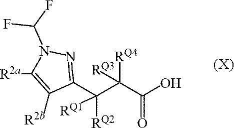
R2^{a} halogen, C₁—C₃fluoroalkyl, C₁—C₃haloalkoxy, C₁—C₃alkoxy, or C₁—C₃alkyl;
R^{2b} is hydrogen, halogen, C₁—C₃fluoroalkyl, C₁—C₃haloalkoxy, C₁—C₃alkoxy, or C₁—C₃alkyl;
R^{Q1} and R^{Q4} are each hydrogen; and
R^{Q2} and R^{Q3} together with the carbon atoms to which they are joined form ring Q, which is an optionally substituted 5-membered thio-lactam ring.

22. The compound of claim 21 wherein ring Q is Q1 or Q2 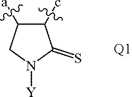 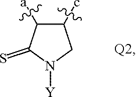
wherein Y is H, methyl or methoxy,
‘a’ denotes the point of attachment to the pyrazole moiety, and ‘c’ denotes the point of attachment to the carboxylate moiety.

23. (canceled)

24. Use of a compound of formula (A) as defined in claim 16 in the manufacture of a herbicide.

25. Use of a compound of formula (B) as defined in claim 17 in the manufacture of a herbicide.

26. Use of a compound of formula (D) as defined in claim 18 in the manufacture of a herbicide.

27. Use of a compound of formula (E) as defined in claim 19 in the manufacture of a herbicide.

28. Use of a compound of formula (J) as defined in claim 20 in the manufacture of a herbicide.

29. Use of a compound of formula (X) as defined in claim 21 in the manufacture of a herbicide.

30. Use of a compound of formula (X) as defined in claim 22 in the manufacture of a herbicide.
